SEQUENCE LISTING (103 sequences)

SEQ ID NO: 1 - protein, 121 aa, synthetic construct
EVQLQQSGPVLVKPGASVKMSCKASGYTFTDYYVNWVKKSHGKSPEWIGIIKPYNGGTSYNQKFKGKATLTVDKSSSTVYMELNSLTSEDSAVYHCARFAYSRNPYAMDYWGQGTSVTVSS

SEQ ID NO: 2 - protein, 112 aa, synthetic construct
DVLLTQTPLSLPVSLGDQASISCRSSQSIVHSTGNTYLEWYLQKPGQSPKVLIYKVSTRFSGVPDRFSGSGSGTDFTLKISRVEAEDLGVYYCFQGSHVPPTFGGGTKLEIK

SEQ ID NO: 3 - protein, 120 aa, synthetic construct
DLQLKESGPGLVKPSQSLSLTCSVTGYSITSGYYWNWIRQLPGNKLEWMGYISFDGSNNYNPSLKTRISITRDTSKNQFFLKLNSVTSEDTATYFCARLRGLLLGHFDVWGTGTTVTVSS

SEQ ID NO: 4 - protein, 107 aa, synthetic construct
DVQMTQSSSYLSVPLGGRVTITCKASDHINNRLAWYQQKPGNAPRLLISGATKLETGIPSRFRGSGSGKDYTLSIFSLQTEDVATYYCQQYWSTSYTFGGGTKLELR

SEQ ID NO: 5 - protein, 120 aa, synthetic construct
DLQLKESGPGLVKPSQSLSLTCSVTGYSITSGYYWNWIRQFPGNKLEWMGFISYDGNNNYNPSLKNRISITRDTSKNQFFLNVKSVTTEDTATYYCSRATGLRKAWFAYWGQGTLVTVSA

SEQ ID NO: 6 - protein, 107 aa, synthetic construct
DIQMTQSSSYLSVSLGGRVTITCKASDHINNWLAWYQQKPGNAPRLLISDTTSLGTGVPSRFRGSGSGKDYTLSITSLQTEDVATYFCQQYWSYPYTFGGGTKLEIR

SEQ ID NO: 7 - protein, 120 aa, synthetic construct
DLQLKESGPGLVKPSQSLSLTCSVTGYSITSGYYWNWIRQFPGNKLEWMGYISYDGSNNYNPSLKTRISITRDTSKNQFFLKLNSVTSEDTATYYCVRERGLLLGHFDVWGTGTTVTVSS

SEQ ID NO: 8 - protein, 107 aa, synthetic construct
DIQMTQSSSYLSVALGGRVTITCKASDHINNRLAWYQQKPGNAPRLLISGATKLETGIPSRFRGSGSGKDYTLSIFSLQTEDVATYYCQQYWSTSYTFGGGTKLELR

SEQ ID NO: 9 - protein, 120 aa, synthetic construct
DLQLKESGPGLVKPSQSLSLTCSVTGYSITSGYYWNWIRQFPGNKLEWMGYISYDGNNNYNPSLKNRISITRDTSKNQFFLNLNSVTTEDTATYYCSRATGLRKAWFAYWGQGTLVTVSA

SEQ ID NO: 10 - protein, 107 aa, synthetic construct; identical to SEQ ID NO: 6

SEQ ID NO: 11 - protein, 120 aa, synthetic construct
DVQLQESGPGLVKPSQSLSLTCSVTGYSITSGYYWNWIRQFPGNKLEWMGYISYDGYNDYNPSLKNRISITRDTSKNQFFLKLNSVTTEDTATYYCARETGLRKAWFAYWGQGTLVTVSG

SEQ ID NO: 12 - protein, 107 aa, synthetic construct
DIQMTQSSSYLSVSLGGRVTITCKASDHINNRLAWYQQKPGNAPRLLISDTSSLETGVPSRFSGSGSGKDYTLRITSLQTEDVATYYCQQYWGTPYTFGGGTKLEIK

SEQ ID NO: 13 - protein, 124 aa, synthetic construct
EVQLVESGGGLVQPKGSLKLSCAASGFSFNTYAMNWVRQAPGKGLKWVARIRTKSNNYATHYADSVKDRFTLSRDDSESMLYLQMNNLKTEDTAMYYCVGGGYGSSASNAMAYWGQGTSVTVSS

SEQ ID NO: 14 - protein, 112 aa, synthetic construct
DIVMTQAAPSVPVTPGESVSISCRSSKSLLHSNGNTYLYWFLQRPGQSPQLLIFRMSNLASGVPDRFSGSGSGTAFTLRISRVEAEDVGVYYCMQHLEYPFTFGSGTKLEIK

SEQ ID NO: 15 - protein, 123 aa, synthetic construct
EVQLVGSGGGLVQPKGSLKLSCAASGFNFNTYVMNWVRQAPGKGLEWVARIRTKSNNYATYYADSVKDRFTISRDDSESKLYLQMNNLKTEDTAMYYCVSGTITRLGGGMDYWGQGTSVTVSS

SEQ ID NO: 16 - protein, 112 aa, synthetic construct
DIVMTQAAPSVPVTPGESVSISCRSSKRLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 17 - protein, 123 aa, synthetic construct
EVQLVGSGGGLVQPKGSLKLSCAASGFNFNTYVMNWVRQAPGKGLEWVARIRTKSNNYATYYADSVKDRFTISRDDSESKLYLQMNNLKTEDTAMYYCVSGTITRLGGGLDYWGQGTSVTVSS

SEQ ID NO: 18 - protein, 112 aa, synthetic construct; identical to SEQ ID NO: 16

SEQ ID NO: 19 - protein, 123 aa, synthetic construct
EVQLVESGGGLVQPKGSLKLSCAASGFSFNTYVMNWVRQAPGKGLNWVARIRTKSNNYATFYVDSVKDRFTISRDDSENMLYLQMNNLKIEDTAIYYCVSGTITRLGAGLDYWGQGTSVTVSS

SEQ ID NO: 20 - protein, 112 aa, synthetic construct
DIVMTQAAPSVPVTPGESVSISCRSSKSLLHSNGNTYLYWFLQRPGQSPQLLIYRMSNLASGVPDRFSGSGSGTAFTLRISRVEAEDVGVYYCMQHLEYPFTFGGGTKLEIK

SEQ ID NO: 21 - protein, 5 aa, synthetic construct
DYYVN

SEQ ID NO: 22 - protein, 6 aa, synthetic construct
SGYYWN

SEQ ID NO: 23 - protein, 5 aa, synthetic construct
TYAMN

SEQ ID NO: 24 - protein, 5 aa, synthetic construct
TYVMN

SEQ ID NO: 25 - protein, 17 aa, synthetic construct
IIKPYNGGTSYNQKFKG

SEQ ID NO: 26 - protein, 16 aa, synthetic construct
YISFDGSNNYNPSLKT

SEQ ID NO: 27 - protein, 16 aa, synthetic construct
FISYDGNNNYNPSLKN

SEQ ID NO: 28 - protein, 16 aa, synthetic construct
YISYDGSNNYNPSLKT

SEQ ID NO: 29 - protein, 16 aa, synthetic construct
YISYDGNNNYNPSLKN

SEQ ID NO: 30 - protein, 16 aa, synthetic construct
YISYDGYNDYNPSLKN

SEQ ID NO: 31 - protein, 19 aa, synthetic construct
RIRTKSNNYATHYADSVKD

SEQ ID NO: 32 - protein, 19 aa, synthetic construct
RIRTKSNNYATYYADSVKD

SEQ ID NO: 33 - protein, 19 aa, synthetic construct
RIRTKSNNYATFYVDSVKD

SEQ ID NO: 34 - protein, 12 aa, synthetic construct
FAYSRNPYAMDY

SEQ ID NO: 35 - protein, 11 aa, synthetic construct
LRGLLLGHFDV

SEQ ID NO: 36 - protein, 11 aa, synthetic construct
ATGLRKAWFAY

SEQ ID NO: 37 - protein, 11 aa, synthetic construct
ERGLLLGHFDV

SEQ ID NO: 38 - protein, 11 aa, synthetic construct
ETGLRKAWFAY

SEQ ID NO: 39 - protein, 13 aa, synthetic construct
GGYGSSASNAMAY

SEQ ID NO: 40 - protein, 12 aa, synthetic construct
GTITRLGGGMDY

SEQ ID NO: 41 - protein, 12 aa, synthetic construct
GTITRLGGGLDY

SEQ ID NO: 42 - protein, 12 aa, synthetic construct
GTITRLGAGLDY

SEQ ID NO: 43 - protein, 16 aa, synthetic construct
RSSQSIVHSTGNTYLE

SEQ ID NO: 44 - protein, 11 aa, synthetic construct
KASDHINNRLA

SEQ ID NO: 45 - protein, 11 aa, synthetic construct
KASDHINNWLA

SEQ ID NO: 46 - protein, 16 aa, synthetic construct
RSSKSLLHSNGNTYLY

SEQ ID NO: 47 - protein, 16 aa, synthetic construct
RSSKRLLHSNGNTYLY

SEQ ID NO: 48 - protein, 7 aa, synthetic construct
KVSTRFS

SEQ ID NO: 49 - protein, 7 aa, synthetic construct
GATKLET

SEQ ID NO: 50 - protein, 7 aa, synthetic construct
DTTSLGT

SEQ ID NO: 51 - protein, 7 aa, synthetic construct
DTSSLET

SEQ ID NO: 52 - protein, 7 aa, synthetic construct
RMSNLAS

SEQ ID NO: 53 - protein, 9 aa, synthetic construct
FQGSHVPPT

SEQ ID NO: 54 - protein, 9 aa, synthetic construct
QQYWSTSYT

SEQ ID NO: 55 - protein, 9 aa, synthetic construct
QQYWSYPYT

SEQ ID NO: 56 - protein, 9 aa, synthetic construct
QQYWGTPYT

SEQ ID NO: 57 - protein, 9 aa, synthetic construct
MQHLEYPFT

SEQ ID NO: 58 - protein, 16 aa, synthetic construct
  VARIANT 1: note = X is F or Y
  VARIANT 4: note = X is F or Y
  VARIANT 6: note = X is G or A
  VARIANT 7: note = X is S, N or Y
  VARIANT 9: note = X is D or N
  VARIANT 16: note = X is N or T
XISXDXXNXYNPSLKX

SEQ ID NO: 59 - protein, 11 aa, synthetic construct
  VARIANT 9: note = X is R or W
KASDHINNXLA

SEQ ID NO: 60 - protein, 9 aa, synthetic construct
  VARIANT 5: note = X is G or S
  VARIANT 6: note = X is T or Y
  VARIANT 7: note = X is P or S
QQYWXXXYT

SEQ ID NO: 61 - protein, 5 aa, synthetic construct
  VARIANT 3: note = X is A or V
TYXMN

SEQ ID NO: 62 - protein, 19 aa, synthetic construct
  VARIANT 12: note = X is F, H or Y
  VARIANT 14: note = X is A or V
  VARIANT 15..16: note = XX is DS, DA or ES
RIRTKSNNYATXYXXXVKD

SEQ ID NO: 63 - protein, 12 aa, synthetic construct
  VARIANT 8: note = X is A or G
  VARIANT 10: note = X is L or M
GTITRLGXGXDY

SEQ ID NO: 64 - protein, 16 aa, synthetic construct
  VARIANT 5: note = X is R or S
  VARIANT 10: note = X is N or Q
  VARIANT 11: note = X is G or A
RSSKXLLHSXXNTYLY

SEQ ID NO: 65 - protein, 17 aa, synthetic construct
IIKPYNAGTSYNQKFKG

SEQ ID NO: 66 - protein, 17 aa, synthetic construct
IIKPYQGGTSYNQKFKG

SEQ ID NO: 67 - protein, 16 aa, synthetic construct
YISFDASNNYNPSLKT

SEQ ID NO: 68 - protein, 16 aa, synthetic construct
FISYDANNNYNPSLKN

SEQ ID NO: 69 - protein, 16 aa, synthetic construct
YISYDASNNYNPSLKT

SEQ ID NO: 70 - protein, 16 aa, synthetic construct
YISYDANNNYNPSLKN

SEQ ID NO: 71 - protein, 16 aa, synthetic construct
YISYDAYNDYNPSLKN

SEQ ID NO: 72 - protein, 16 aa, synthetic construct
RSSKSLLHSNANTYLY

SEQ ID NO: 73 - protein, 16 aa, synthetic construct
RSSKSLLHSQGNTYLY

SEQ ID NO: 74 - protein, 16 aa, synthetic construct
RSSKRLLHSNANTYLY

SEQ ID NO: 75 - protein, 16 aa, synthetic construct
RSSKRLLHSQGNTYLY

SEQ ID NO: 76 - protein, 123 aa, synthetic construct
EVQLVESGGGLVQPGGSLRLSCAASGFNFNTYVMNWVRQAPGKGLEWVGRIRTKSNNYATYYADSVKDRFTISRDDSKNSLYLQMNSLKTEDTAVYYCARGTITRLGGGLDYWGQGTLVTVSS

SEQ ID NO: 77 - protein, 123 aa, synthetic construct
EVQLVESGGGLVQPGGSLRLSCAASGFNFNTYVMNWVRQAPGKGLEWVARIRTKSNNYATYYADSVKDRFTISRDDSKNSLYLQMNSLKTEDTAVYYCVSGTITRLGGGLDYWGQGTLVTVSS

SEQ ID NO: 78 - protein, 123 aa, synthetic construct
EVQLVESGGGLVQPGGSLRLSCAASGFNFNTYVMNWVRQAPGKGLEWVARIRTKSNNYATYYADSVKDRFTISRDDSKSSLYLQMNSLKTEDTAVYYCVSGTITRLGGGLDYWGQGTLVTVSS

SEQ ID NO: 79 - protein, 123 aa, synthetic construct
EVQLVESGGGLVQPGGSLRLSCAASGFNFNTYVMNWVRQAPGKGLEWVARIRTKSNNYATYYADSVKDRFTISRDDSESSLYLQMNSLKTEDTAMYYCVSGTITRLGGGLDYWGQGTLVTVSS

SEQ ID NO: 80 - protein, 112 aa, synthetic construct
DIVMTQSPLSLPVTPGEPASISCRSSKRLLHSNGNTYLYWYLQKPGQSPQLLIYRMSNLASGVPDRFSGSGSGTDFTLKISRVEAEDVGVYYCMQHLEYPFTFGGGTKLEIK

SEQ ID NO: 81 - protein, 112 aa, synthetic construct
DIVMTQSPLSLPVTPGEPASISCRSSKRLLHSNGNTYLYWFLQKPGQSPQLLIYRMSNLASGVPDRFSGSGSGTDFTLKISRVEAEDVGVYYCMQHLEYPFTFGGGTKLEIK

SEQ ID NO: 82 - protein, 112 aa, synthetic construct
DIVMTQSPLSLPVTPGEPASISCRSSKRLLHSNGNTYLYWYLQKPGQSPQLLIYRMSNLASGVPDRFSGSGSGTAFTLKISRVEAEDVGVYYCMQHLEYPFTFGGGTKLEIK

SEQ ID NO: 83 - protein, 112 aa, synthetic construct
DIVMTQSPLSLPVTPGEPASISCRSSKRLLHSNGNTYLYWFLQKPGQSPQLLIYRMSNLASGVPDRFSGSGSGTAFTLKISRVEAEDVGVYYCMQHLEYPFTFGGGTKLEIK

SEQ ID NO: 84 - protein, 123 aa, synthetic construct
EVQLVESGGGLVQPGGSLRLSCAASGFSFNTYVMNWVRQAPGKGLEWVGRIRTKSNNYATFYVDSVKDRFTISRDDSKNSLYLQMNSLKTEDTAVYYCARGTITRLGAGLDYWGQGTLVTVSS

SEQ ID NO: 85 - protein, 123 aa, synthetic construct
EVQLVESGGGLVQPGGSLRLSCAASGFSFNTYVMNWVRQAPGKGLEWVARIRTKSNNYATFYVDSVKDRFTISRDDSKNSLYLQMNSLKTEDTAVYYCVSGTITRLGAGLDYWGQGTLVTVSS

SEQ ID NO: 86 - protein, 123 aa, synthetic construct
EVQLVESGGGLVQPGGSLRLSCAASGFSFNTYVMNWVRQAPGKGLNWVARIRTKSNNYATFYVDSVKDRFTISRDDSKNSLYLQMNSLKTEDTAVYYCVSGTITRLGAGLDYWGQGTLVTVSS

SEQ ID NO: 87 - protein, 123 aa, synthetic construct
EVQLVESGGGLVQPGGSLRLSCAASGFSFNTYVMNWVRQAPGKGLNWVARIRTKSNNYATFYVDSVKDRFTISRDDSENSLYLQMNSLKTEDTAVYYCVSGTITRLGAGLDYWGQGTLVTVSS

SEQ ID NO: 88 - protein, 112 aa, synthetic construct
DIVMTQSPLSLPVTPGEPASISCRSSKSLLHSNGNTYLYWYLQKPGQSPQLLIYRMSNLASGVPDRFSGSGSGTDFTLKISRVEAEDVGVYYCMQHLEYPFTFGGGTKLEIK

SEQ ID NO: 89 - protein, 112 aa, synthetic construct
DIVMTQSPLSLPVTPGEPASISCRSSKSLLHSNGNTYLYWFLQKPGQSPQLLIYRMSNLASGVPDRFSGSGSGTDFTLKISRVEAEDVGVYYCMQHLEYPFTFGGGTKLEIK

SEQ ID NO: 90 - protein, 112 aa, synthetic construct
DIVMTQSPLSLPVTPGEPASISCRSSKSLLHSNGNTYLYWYLQKPGQSPQLLIYRMSNLASGVPDRFSGSGSGTAFTLKISRVEAEDVGVYYCMQHLEYPFTFGGGTKLEIK

SEQ ID NO: 91 - protein, 112 aa, synthetic construct
DIVMTQSPLSLPVTPGEPASISCRSSKSLLHSNGNTYLYWFLQKPGQSPQLLIYRMSNLASGVPDRFSGSGSGTAFTLKISRVEAEDVGVYYCMQHLEYPFTFGGGTKLEIK

SEQ ID NO: 92 - protein, 112 aa, synthetic construct
DIVMTQSPLSLPVTPGEPASISCRSSKRLLHSNANTYLYWYLQKPGQSPQLLIYRMSNLASGVPDRFSGSGSGTDFTLKISRVEAEDVGVYYCMQHLEYPFTFGGGTKLEIK

SEQ ID NO: 93 - protein, 112 aa, synthetic construct
DIVMTQSPLSLPVTPGEPASISCRSSKRLLHSNANTYLYWFLQKPGQSPQLLIYRMSNLASGVPDRFSGSGSGTDFTLKISRVEAEDVGVYYCMQHLEYPFTFGGGTKLEIK

SEQ ID NO: 94 - protein, 112 aa, synthetic construct
DIVMTQSPLSLPVTPGEPASISCRSSKRLLHSNANTYLYWYLQKPGQSPQLLIYRMSNLASGVPDRFSGSGSGTAFTLKISRVEAEDVGVYYCMQHLEYPFTFGGGTKLEIK

SEQ ID NO: 95 - protein, 112 aa, synthetic construct
DIVMTQSPLSLPVTPGEPASISCRSSKRLLHSNANTYLYWFLQKPGQSPQLLIYRMSNLASGVPDRFSGSGSGTAFTLKISRVEAEDVGVYYCMQHLEYPFTFGGGTKLEIK

SEQ ID NO: 96 - protein, 112 aa, synthetic construct
DIVMTQSPLSLPVTPGEPASISCRSSKSLLHSNANTYLYWYLQKPGQSPQLLIYRMSNLASGVPDRFSGSGSGTDFTLKISRVEAEDVGVYYCMQHLEYPFTFGGGTKLEIK

SEQ ID NO: 97 - protein, 112 aa, synthetic construct
DIVMTQSPLSLPVTPGEPASISCRSSKSLLHSNANTYLYWFLQKPGQSPQLLIYRMSNLASGVPDRFSGSGSGTDFTLKISRVEAEDVGVYYCMQHLEYPFTFGGGTKLEIK

SEQ ID NO: 98 - protein, 112 aa, synthetic construct
DIVMTQSPLSLPVTPGEPASISCRSSKSLLHSNANTYLYWYLQKPGQSPQLLIYRMSNLASGVPDRFSGSGSGTAFTLKISRVEAEDVGVYYCMQHLEYPFTFGGGTKLEIK

SEQ ID NO: 99 - protein, 112 aa, synthetic construct
DIVMTQSPLSLPVTPGEPASISCRSSKSLLHSNANTYLYWFLQKPGQSPQLLIYRMSNLASGVPDRFSGSGSGTAFTLKISRVEAEDVGVYYCMQHLEYPFTFGGGTKLEIK

SEQ ID NO: 100 - protein, 19 aa, synthetic construct
RIRTKSNNYATYYADAVKD

SEQ ID NO: 101 - protein, 19 aa, synthetic construct
RIRTKSNNYATYYAESVKD

SEQ ID NO: 102 - protein, 19 aa, synthetic construct
RIRTKSNNYATFYVDAVKD

SEQ ID NO: 103 - protein, 19 aa, synthetic construct
RIRTKSNNYATFYVESVKD

(19) United States
(12) Patent Application Publication
(10) Pub. No.: US 2025/0368744 A1
(43) Pub. Date: Dec. 4, 2025

(54) ANTI-CCR8 ANTIBODIES AND USES THEREOF

(71) Applicant: CONCEPT TO MEDICINE BIOTECH CO., LTD., Shanghai (CN)

(72) Inventors: Yuanyuan YANG, Shanghai (CN); Yuehua CHEN, Shanghai (CN); Wenqing JIANG, Shanghai (CN); Feifei CUI, Shangahi (CN); Lei FANG, Shanghai (CN)

(21) Appl. No.: 18/723,322

(22) PCT Filed: Dec. 23, 2022
(86) PCT No.: PCT/CN2022/141410

§ 371 Date: Jun. 21, 2024

(30) Foreign Application Priority Data
Dec. 23, 2021 (WO) PCT/CN2021/140992
Oct. 21, 2022 (WO) PCT/CN2022/126704

(51) Int. Cl. C07K 16/28; A61K 39/00; A61P 35/00

(52) U.S. Cl. CPC C07K 16/2866; A61P 35/00; A61K 2039/505; C07K 2317/24; C07K 2317/52; C07K 2317/732; C07K 2317/92

(57) ABSTRACT

Provided are anti-CCR8 antibodies, including murine antibodies, humanized antibodies, and those with further optimized CDR sequences. Antibodies exhibited high antagonist and ADCC activities and can be used to treat cancers and inflammatory conditions.

### CROSS REFERENCE TO RELATED APPLICATIONS

The present application is a U.S. National Stage of PCT International Patent Application No. PCT/CN2022/141410 filed Dec. 23, 2022, which claims priority of PCT International Patent Application No. PCT/CN2022/126704 filed Oct. 21, 2022, and PCT International Patent Application No. PCT/CN2021/140992 filed Dec. 23, 2021, each of which is incorporated herein by reference in its entirety.

### REFERENCE TO AN ELECTRONIC SEQUENCE LISTING

The contents of the electronic sequence listing “344057” size 102,235 bytes, and Date of Creation Jun. 19, 2024, is herein incorporated by reference in its entirety.

### BACKGROUND

The C-C motif chemokine receptor 8 (CCR8) is a seven-transmembrane G-protein coupled receptor (GPCR). CCR8 is preferentially expressed in the thymus. CCR8 and its ligands play important roles in regulation of monocyte chemotaxis and thymic cell apoptosis. More specifically, CCR8 may contribute to the proper positioning of activated T cells within the antigenic challenge sites and specialized areas of lymphoid tissues.

CCR8 is selectively expressed on T helper type 2 (Th2) cells but not on T helper type 1 (Th1) cells. Th2 cells play an important role in the allergic inflammatory response which occurs at sites of allergen exposure. A ligand for CCR8 is the CC chemokine CCL1, which is a chemoattractant for Th2 cells. The CCR8/CCL1 receptor/ligand pair may therefore play a role in development of allergic inflammation conditions such as asthma, atopic dermatitis and allergic rhinitis. This role includes recruitment of Th2 cells to sites of allergic inflammation, the production of Th2 cytokines at those sites, and the subsequent mobilization of eosinophils and basophils.

CCR8-knockout mice showed impaired Th2 immune responses in models of allergic inflammation. In ovalbumin- and cockroach antigen-induced allergic pulmonary inflammation, the levels of Th2 cytokines (IL-4, IL-5 and IL-13) and the number of recruited eosinophils were significantly reduced in lungs of the CCR8 knockout mice. Inhibition of CCR8, therefore, can be useful for ameliorating symptoms of allergic inflammatory conditions, such as asthma, atopic dermatitis, and allergic rhinitis.

CCR8 has also been identified as a marker for tumor-infiltrating Tregs, as CCR8 expression is selectively upregulated in these Tregs in multiple cancers, including breast, colorectal, and lung. These CCR8+ Tregs represent a highly activated and suppressive subpopulation of Tregs, and high abundance of CCR8+ Tregs in these tumor types is associated with poor prognosis. Therefore, CCR8 is a promising therapeutic target to deplete tumor resident Tregs to augment anti-tumor immunity.

Chemokine receptors, however, have traditionally been very difficult antigens to develop antibodies against. They have low expression on the cell surface and are not very accessible to antibody binding. Also, antibodies generated against peptides corresponding to extracellular domains of chemokine receptors often fail to recognize the intact receptor on the cell, probably because of differences in secondary structure.

CCR8 is an extremely challenging GPCR for antibody development, with limited success in generating a cross species-reactive antibodies in the past (cynomolgus and human). Furthermore, it is even more challenging to find functional antibodies for a GPCR, including CCR8.

### SUMMARY

The present disclosure, in various embodiments, provides antibodies and antigen-binding fragments specific to the human CCR8 protein. Experimental testing shows that these newly identified antibodies can bind to the human CCR8 protein potently and specifically, and most can cross-react with the cynomolgus CCR8 protein, which are different from most of the benchmark's antibodies and makes it feasible to demonstrate the pre-clinical safety in non-human primates. Moreover, in vitro studies have demonstrated that most of these antibodies could specifically block CCR8 signaling induced by its ligand CCL1. Furthermore, when used with constant regions with full or enhanced Fc function, these antibodies could induce antibody-dependent cell-mediated cytotoxicity (ADCC) of target cells expressing CCR8. In vivo experiments have shown that these antibodies, especially when used with constant regions with enhanced Fc function, effectively inhibited tumor growth.

In accordance with one embodiment of the present disclosure, provided is an antibody or antigen-binding fragment thereof which has specificity to the human C-C motif chemokine receptor 8 (CCR8) protein and comprises a heavy chain variable region (VH) comprising a VH CDR1, a VH CDR2 and a VH CDR3, and a light chain variable region (VL) comprising a VL CDR1, a VL CDR2, and a VL CDR3.

In one embodiment, the VH CDR1 comprises the amino acid sequence of SEQ ID NO: 61; the VH CDR2 comprises the amino acid sequence of SEQ ID NO: 62; the VH CDR3 comprises the amino acid sequence of SEQ ID NO: 63 or 39; the VL CDR1 comprises the amino acid sequence of SEQ ID NO: 64; the VL CDR2 comprises the amino acid sequence of SEQ ID NO: 52; and the VL CDR3 comprises the amino acid sequence of SEQ ID NO: 57.

In another embodiment, the VH CDR1 comprises the amino acid sequence of SEQ ID NO: 22; the VH CDR2 comprises the amino acid sequence of SEQ ID NO: 58; the VH CDR3 comprises the amino acid sequence of SEQ ID NO: 35, 36, 37 or 38; the VL CDR1 comprises the amino acid sequence of SEQ ID NO: 59; the VL CDR2 comprises the amino acid sequence of SEQ ID NO: 49, 50 or 51; and the VL CDR3 comprises the amino acid sequence of SEQ ID NO: 60

In another embodiment, the VH CDR1 comprises the amino acid sequence of SEQ ID NO: 21; the VH CDR2 comprises the amino acid sequence of SEQ ID NO: 25, 65 or 66; the VH CDR3 comprises the amino acid sequence of SEQ ID NO: 34; the VL CDR1 comprises the amino acid sequence of SEQ ID NO: 43; the VL CDR2 comprises the amino acid sequence of SEQ ID NO: 48; and the VL CDR3 comprises the amino acid sequence of SEQ ID NO: 53.

In some embodiments, the antibody or fragment is humanized. In some embodiments, the antibody or fragment comprises an Fc fragment with enhanced antibody dependent cellular cytotoxicity (ADCC). An example Fc fragment is a human IgG1 fragment with one or more substitutions selected from the group consisting of L234Y, L235Q, G236W, S239D/M, F243L, H268D, D270E, R292P, S298A, Y300L, V305I, K326D, A330L/M, I332E, K334A/E, P396L, according to EU numbering.

Also provided are multispecific antibodies comprising an antigen-binding fragment of the present disclosure and one or more antibody or antigen-binding fragment having binding specificity to a target antigen that is not CCR8.

Also provided, in another embodiment, is a chimeric antigen receptor (CAR) comprising an antigen-binding fragment of the present disclosure, a transmembrane domain, a costimulatory domain, and a CD3 ξ intracellular domain.

Polynucleotides are also provided, encoding the antibody or antigen-binding fragment thereof or the CAR of the present disclosure. In some embodiments, the polynucleotide is mRNA, which is optionally chemically modified.

Methods and uses for treating cancer and inflammatory conditions are also provided, with the antibody or antigen-binding fragment thereof of the present disclosure.

### BRIEF DESCRIPTION OF THE DRAWINGS

FIG. 1 shows the binding affinity of the identified antibodies to human CCR8, cyno CCR8 and human CCR4.

FIG. 2 shows the blocking activities of tested chimeric antibodies.

FIG. 3 shows the ADCC signaling activities of tested chimeric antibodies.

FIG. 4 shows the natural killer cell mediated ADCC killing effect induced by tested antibodies.

FIG. 5 shows the in vivo tumor inhibition activities of tested antibodies.

FIG. 6 shows the binding of humanized antibodies to human CCR8, cyno CCR8.

FIG. 7 shows the ADCC activity of tested humanized CCR8 antibodies.

FIG. 8 shows the blocking activity of tested humanized CCR8 antibodies.

FIG. 9 shows the binding and functional property of anti-CCR8 chimeric antibodies after removal of potential PTM site.

FIG. 10 shows the binding and functional property of humanized CCR8 antibodies after removal of potential PTM site.

FIG. 11 shows the in vivo tumor inhibition activities of humanized CCR8 antibodies after removal of potential PTM site.

FIG. 12 shows in vitro comparison of Hu200C9B9-7 NG-NA and benchmark antibodies.

FIG. 13 shows in vivo and ex vivo comparison of Hu200C9B9-7 NG-NA and benchmark antibodies.

FIG. 14 shows the ADCC activity of Hu200C9B9-7 NG-NA with different Fc engineering.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)2, F(ab)2, Fab′, Fab, Fv, scFv and the like. Regardless of structure, an antibody fragment binds with the same antigen that is recognized by the intact antibody. The term “antibody fragment” includes aptamers, spiegeleisen, and diabodies. The term “antibody fragment” also includes any synthetic or genetically engineered protein that acts like an antibody by binding to a specific antigen to form a complex.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively.

The immunoglobulin subclasses (isotypes) e.g., IgG1, IgG2, IgG3, IgG4, IgG5, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000 Daltons. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′) 2, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-Id antibodies to LIGHT antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

As used herein, the term “chimeric antibody” will be held to mean any antibody wherein the immunoreactive region or site is obtained or derived from a first species and the constant region (which may be intact, partial or modified in accordance with the instant disclosure) is obtained from a second species. In certain embodiments the target binding region or site will be from a non-human source (e.g. mouse or primate) and the constant region is human.

Antibodies disclosed herein can be from any animal origin including birds and mammals. Preferably, the antibodies are human, murine, donkey, rabbit, goat, guinea pig, camel, llama, horse, or chicken antibodies. In some embodiments, the variable region may be condricthoid in origin (e.g., from sharks).

As used herein, the term “recombinant” as it pertains to polypeptides or polynucleotides intends a form of the polypeptide or polynucleotide that does not exist naturally, a non-limiting example of which can be created by combining polynucleotides that would not normally occur together.

Hybridoma technology can be performed under conditions of different “stringency”. In general, a low stringency hybridization reaction is carried out at about 40° C. in about 10×SSC or a solution of equivalent ionic strength/temperature. A moderate stringency hybridization is typically performed at about 50° C. in about 6×SSC, and a high stringency hybridization reaction is generally performed at about 60° C. in about 1×SSC. Hybridization reactions can also be performed under “physiological conditions” which is well known to one of skill in the art. A nonlimiting example of a physiological condition is the temperature, ionic strength, pH and concentration of Mg2+ normally found in a cell.

### Anti-CCR8 Antibodies

As demonstrated in the appended experimental examples, the instant inventors were able to generate anti-CCR8 antibodies 84D1-2H3, 86D4E12A5, 96G3-1F10, 99D1-1E11, 101D5G10G4, 115C5E3B8, 163H9D5, 187B5F10, 195H8D10 and 200C9B9 (Table 1) all of which have high binding affinity to the human CCR8 protein. The binding is specific as they did not bind to CCR4. All of these antibodies, except 163H9D5, could also cross-react with the cyno CCR8 protein, facilitating preclinical development of these antibodies.

Moreover, all of these antibodies, except 84D1-2H3, exhibited strong antagonist activities. Regardless their antagonist activities, however, all the antibodies could induce ADCC and were able to inhibit tumor growth in the animal model.

Based on the sequences, these antibodies can be classified into three groups. As shown in Table 1A-B, Group B includes 163H9D5, 187B5F10, 195H8D10 and 200C9B9; Group A includes 86D4E12A5, 96G3-1F10, 99D1-1E11, 101D5G10G4 and 115C5E3B8; and Group C includes 84D1-2H3. Each group of antibodies share high homology among each CDR, which are therefore interchangeable.

In accordance with one embodiment of the present disclosure, provided is an antibody or antigen-binding fragment thereof. In some embodiments, the antibody or antigen-binding fragment thereof has binding specificity to the human CCR8 protein. In some embodiments, the antibody or antigen-binding fragment thereof includes a heavy chain variable region (VH) that includes a VH CDR1, a VH CDR2 and a VH CDR3, and a light chain variable region (VL) that includes a VL CDR1, a VL CDR2, and a VL CDR3.

In some embodiments, in relation to antibody Group B, the VH CDR1 includes the amino acid sequence of SEQ ID NO: 61, the VH CDR2 includes the amino acid sequence of SEQ ID NO: 62, the VH CDR3 includes the amino acid sequence of SEQ ID NO: 63 or 39, the VL CDR1 includes the amino acid sequence of SEQ ID NO: 64, the VL CDR2 includes the amino acid sequence of SEQ ID NO: 52, and the VL CDR3 includes the amino acid sequence of SEQ ID NO: 57.

As shown in Table 1B, SEQ ID NO: 61 has the sequence of TYXMN, where X is A or V. SEQ ID NO: 62 has the sequence of RIRTKSNNYATX₁YX₂X₃X₄VKD, where X₁ is F, H or Y, X₂ is A or V and X₃X₄ is DS, DA or ES. SEQ ID NO: 63 has the sequence of GTITRLGX₁GX₂DY, where X₁ is A or G, and X₂ is L or M. SEQ ID NO: 64 has the sequence of RSSKX₁LLHSX₂X₃NTYLY, wherein X₁ is R or S, X₂ is N or Q, and X₃ is G or A.

In some embodiments, the VH CDR1 includes the amino acid sequence of SEQ ID NO: 61, the VH CDR2 includes the amino acid sequence of SEQ ID NO: 62, the VH CDR3 includes the amino acid sequence of SEQ ID NO: 63, the VL CDR1 includes the amino acid sequence of SEQ ID NO: 64, the VL CDR2 includes the amino acid sequence of SEQ ID NO: 52, and the VL CDR3 includes the amino acid sequence of SEQ ID NO: 57.

In some embodiments, the VH CDR1 includes the amino acid sequence of SEQ ID NO: 61, the VH CDR2 includes the amino acid sequence of SEQ ID NO: 62, the VH CDR3 includes the amino acid sequence of SEQ ID NO: 39, the VL CDR1 includes the amino acid sequence of SEQ ID NO: 64, the VL CDR2 includes the amino acid sequence of SEQ ID NO: 52, and the VL CDR3 includes the amino acid sequence of SEQ ID NO: 57.

In some embodiments, the VH CDR1 includes the amino acid sequence of SEQ ID NO: 23 or 24, the VH CDR2 includes the amino acid sequence of SEQ ID NO: 31, 32, 33, 100, 101, 102, or 103, the VH CDR3 includes the amino acid sequence of SEQ ID NO: 39, 40, 41 or 42, the VL CDR1 includes the amino acid sequence of SEQ ID NO: 46 or 47, the VL CDR2 includes the amino acid sequence of SEQ ID NO: 52, and the VL CDR3 includes the amino acid sequence of SEQ ID NO: 57.

Sequence analysis revealed that the VL CDR1 sequences (SEQ ID NO: 46 and 47) include residues that can potentially be modified post-translationally. To avoid post-translational modification (PTM) risks and thus simplify manufacturing, the instant disclosure designed and tested certain de-risked versions of the VL CDR1, including SEQ ID NO: 72, 73, 74 and 75. In some embodiments, therefore, provided are antibodies and fragments having CDR sequences in which the VH CDR1 includes the amino acid sequence of SEQ ID NO: 23 or 24, the VH CDR2 includes the amino acid sequence of SEQ ID NO: 31, 32, 33, 100, 101, 102, or 103, the VH CDR3 includes the amino acid sequence of SEQ ID NO: 39, 40, 41 or 42, the VL CDR1 includes the amino acid sequence of SEQ ID NO: 46, 47, 72, 73, 74, or 75, the VL CDR2 includes the amino acid sequence of SEQ ID NO: 52, and the VL CDR3 includes the amino acid sequence of SEQ ID NO: 57.

In some embodiments, in relation to antibodies of Group A, the VH CDR1 includes the amino acid sequence of SEQ ID NO: 22, the VH CDR2 includes the amino acid sequence of SEQ ID NO: 58, the VH CDR3 includes the amino acid sequence of SEQ ID NO: 35, 36, 37 or 38, the VL CDR1 includes the amino acid sequence of SEQ ID NO: 59, the VL CDR2 includes the amino acid sequence of SEQ ID NO: 49, 50 or 51, and the VL CDR3 includes the amino acid sequence of SEQ ID NO: 60.

As shown in Table 1B, SEQ ID NO: 58 has the sequence of X₁ISX₂DX₃X₄NXSYNPSLKX₆, where X₁ is F or Y, X₂ is F or Y, X₃ is G or A, X₄ is S, N or Y, X₅ is D or N, and X₆ is N or T. SEQ ID NO: 59 has the sequence of KASDHINNXLA, where X is R or W. SEQ ID NO: 60 has the sequence of QQYWX₁X₂X₃YT, where X₁ is G or S, X₂ is T or Y, and X₃ is P or S.

In some embodiments, the VH CDR1 includes the amino acid sequence of SEQ ID NO: 22, the VH CDR2 includes the amino acid sequence of SEQ ID NO: 26, 27, 28, 29 or 30, the VH CDR3 includes the amino acid sequence of SEQ ID NO: 35, 36, 37 or 38, the VL CDR1 includes the amino acid sequence of SEQ ID NO: 44 or 45, the VL CDR2 includes the amino acid sequence of SEQ ID NO: 49, 50 or 51, and the VL CDR3 includes the amino acid sequence of SEQ ID NO: 54, 55 or 56.

In some embodiments, the VH CDR2 can be PTM de-risked, such as those provided in SEQ ID NO: 67-71. In some embodiments, the VH CDR1 includes the amino acid sequence of SEQ ID NO: 22, the VH CDR2 includes the amino acid sequence of SEQ ID NO: 26, 27, 28, 29, 30, 67, 68, 69, 70 or 71, the VH CDR3 includes the amino acid sequence of SEQ ID NO: 35, 36, 37 or 38, the VL CDR1 includes the amino acid sequence of SEQ ID NO: 44 or 45, the VL CDR2 includes the amino acid sequence of SEQ ID NO: 49, 50 or 51, and the VL CDR3 includes the amino acid sequence of SEQ ID NO: 54, 55 or 56.

In some embodiments, provided is an antibody or antigen-binding fragment that is derived from antibody 84D1-2H3. In some embodiments, the VH CDR1 includes the amino acid sequence of SEQ ID NO: 21; the VH CDR2 includes the amino acid sequence of SEQ ID NO: 25; the VH CDR3 includes an amino acid sequence selected from the group consisting of SEQ ID NO: 34; the VL CDR1 includes the amino acid sequence of SEQ ID NO: 43; the VL CDR2 includes the amino acid sequence of SEQ ID NO: 48; and the VL CDR3 includes an amino acid sequence selected from the group consisting SEQ ID NO: 53.

In some embodiments, the VH CDR2 is PTM de-risked. In some embodiments, the VH CDR1 includes the amino acid sequence of SEQ ID NO: 21; the VH CDR2 includes the amino acid sequence of SEQ ID NO: 25, 65 or 66; the VH CDR3 includes an amino acid sequence selected from the group consisting of SEQ ID NO: 34; the VL CDR1 includes the amino acid sequence of SEQ ID NO: 43; the VL CDR2 includes the amino acid sequence of SEQ ID NO: 48; and the VL CDR3 includes an amino acid sequence selected from the group consisting SEQ ID NO: 53.

In some embodiments, the VH includes the amino acid sequence of SEQ ID NO: 1 or a sequence having at least 75%, 80%, 85%, 90%, 95% or 99% sequence identity to SEQ ID NO: 1, while retaining the VH CDRs of SEQ ID NO: 1 or PTM re-risked versions thereof. In some embodiments, the VL includes the amino acid sequence of SEQ ID NO: 2 or a sequence having at least 75%, 80%, 85%, 90%, 95% or 99% sequence identity to SEQ ID NO: 2, while retaining the VL CDRs of SEQ ID NO: 2 or PTM re-risked versions thereof. In some embodiments, the VH includes the amino acid sequence of SEQ ID NO: 1, and the VL includes the amino acid sequence of SEQ ID NO: 2.

Also provided, in some embodiments, are antibodies and antigen-binding fragments therefore that bind to the same epitope on CCR8 as 84D1-2H3. Also provided, in some embodiments, are antibodies and antigen-binding fragments therefore that competes with 84D1-2H3 in binding to CCR8.

In some embodiments, provided is an antibody or antigen-binding fragment that is derived from antibody 86D4E12A5. In some embodiments, the VH CDR1 includes the amino acid sequence of SEQ ID NO: 22; the VH CDR2 includes the amino acid sequence of SEQ ID NO: 26; the VH CDR3 includes an amino acid sequence selected from the group consisting of SEQ ID NO: 35; the VL CDR1 includes the amino acid sequence of SEQ ID NO: 44; the VL CDR2 includes the amino acid sequence of SEQ ID NO: 49; and the VL CDR3 includes an amino acid sequence selected from the group consisting SEQ ID NO: 54.

In some embodiments, the VH CDR2 is PTM de-risked. In some embodiments, the VH CDR1 includes the amino acid sequence of SEQ ID NO: 22; the VH CDR2 includes the amino acid sequence of SEQ ID NO: 26 or 67; the VH CDR3 includes an amino acid sequence selected from the group consisting of SEQ ID NO: 35; the VL CDR1 includes the amino acid sequence of SEQ ID NO: 44; the VL CDR2 includes the amino acid sequence of SEQ ID NO: 49; and the VL CDR3 includes an amino acid sequence selected from the group consisting SEQ ID NO: 54.

In some embodiments, the VH includes the amino acid sequence of SEQ ID NO: 3 or a sequence having at least 75%, 80%, 85%, 90%, 95% or 99% sequence identity to SEQ ID NO: 3, while retaining the VH CDRs of SEQ ID NO: 3 or PTM re-risked versions thereof. In some embodiments, the VL includes the amino acid sequence of SEQ ID NO: 4 or a sequence having at least 75%, 80%, 85%, 90%, 95% or 99% sequence identity to SEQ ID NO: 4, while retaining the VL CDRs of SEQ ID NO: 4 or PTM re-risked versions thereof. In some embodiments, the VH includes the amino acid sequence of SEQ ID NO: 3, and the VL includes the amino acid sequence of SEQ ID NO: 4.

Also provided, in some embodiments, are antibodies and antigen-binding fragments therefore that bind to the same epitope on CCR8 as 86D4E12A5. Also provided, in some embodiments, are antibodies and antigen-binding fragments therefore that compete with 86D4E12A5 in binding to CCR8.

In some embodiments, provided is an antibody or antigen-binding fragment that is derived from antibody 96G3-1F10. In some embodiments, the VH CDR1 includes the amino acid sequence of SEQ ID NO: 22; the VH CDR2 includes the amino acid sequence of SEQ ID NO: 27; the VH CDR3 includes an amino acid sequence selected from the group consisting of SEQ ID NO: 36; the VL CDR1 includes the amino acid sequence of SEQ ID NO: 45; the VL CDR2 includes the amino acid sequence of SEQ ID NO: 50; and the VL CDR3 includes an amino acid sequence selected from the group consisting SEQ ID NO: 55.

In some embodiments, the VH CDR2 is PTM de-risked. In some embodiments, the VH CDR1 includes the amino acid sequence of SEQ ID NO: 22; the VH CDR2 includes the amino acid sequence of SEQ ID NO: 27 or 68; the VH CDR3 includes an amino acid sequence selected from the group consisting of SEQ ID NO: 36; the VL CDR1 includes the amino acid sequence of SEQ ID NO: 45; the VL CDR2 includes the amino acid sequence of SEQ ID NO: 50; and the VL CDR3 includes an amino acid sequence selected from the group consisting SEQ ID NO: 55.

In some embodiments, the VH includes the amino acid sequence of SEQ ID NO: 5 or a sequence having at least 75%, 80%, 85%, 90%, 95% or 99% sequence identity to SEQ ID NO: 5, while retaining the VH CDRs of SEQ ID NO: 5 or PTM re-risked versions thereof. In some embodiments, the VL includes the amino acid sequence of SEQ ID NO: 6 or a sequence having at least 75%, 80%, 85%, 90%, 95% or 99% sequence identity to SEQ ID NO: 6, while retaining the VL CDRs of SEQ ID NO: 6 or PTM re-risked versions thereof. In some embodiments, the VH includes the amino acid sequence of SEQ ID NO: 5, and the VL includes the amino acid sequence of SEQ ID NO: 6.

Also provided, in some embodiments, are antibodies and antigen-binding fragments therefore that bind to the same epitope on CCR8 as 96G3-1F10. Also provided, in some embodiments, are antibodies and antigen-binding fragments therefore that compete with 96G3-1F10 in binding to CCR8.

In some embodiments, provided is an antibody or antigen-binding fragment that is derived from antibody 99D1-1E11. In some embodiments, the VH CDR1 includes the amino acid sequence of SEQ ID NO: 22; the VH CDR2 includes the amino acid sequence of SEQ ID NO: 28; the VH CDR3 includes an amino acid sequence selected from the group consisting of SEQ ID NO: 37; the VL CDR1 includes the amino acid sequence of SEQ ID NO: 44; the VL CDR2 includes the amino acid sequence of SEQ ID NO: 49; and the VL CDR3 includes an amino acid sequence selected from the group consisting SEQ ID NO: 54.

In some embodiments, the VH CDR2 is PTM de-risked. In some embodiments, the VH CDR1 includes the amino acid sequence of SEQ ID NO: 22; the VH CDR2 includes the amino acid sequence of SEQ ID NO: 28 or 69; the VH CDR3 includes an amino acid sequence selected from the group consisting of SEQ ID NO: 37; the VL CDR1 includes the amino acid sequence of SEQ ID NO: 44; the VL CDR2 includes the amino acid sequence of SEQ ID NO: 49; and the VL CDR3 includes an amino acid sequence selected from the group consisting SEQ ID NO: 54.

In some embodiments, the VH includes the amino acid sequence of SEQ ID NO: 7 or a sequence having at least 75%, 80%, 85%, 90%, 95% or 99% sequence identity to SEQ ID NO: 7, while retaining the VH CDRs of SEQ ID NO: 7 or PTM re-risked versions thereof. In some embodiments, the VL includes the amino acid sequence of SEQ ID NO: 8 or a sequence having at least 75%, 80%, 85%, 90%, 95% or 99% sequence identity to SEQ ID NO: 8, while retaining the VL CDRs of SEQ ID NO: 8 or PTM re-risked versions thereof. In some embodiments, the VH includes the amino acid sequence of SEQ ID NO: 7, and the VL includes the amino acid sequence of SEQ ID NO: 8.

Also provided, in some embodiments, are antibodies and antigen-binding fragments therefore that bind to the same epitope on CCR8 as 99D1-1E11. Also provided, in some embodiments, are antibodies and antigen-binding fragments therefore that compete with 99D1-1E11 in binding to CCR8.

In some embodiments, provided is an antibody or antigen-binding fragment that is derived from antibody 101D5G10G4. In some embodiments, the VH CDR1 includes the amino acid sequence of SEQ ID NO: 22; the VH CDR2 includes the amino acid sequence of SEQ ID NO: 29; the VH CDR3 includes an amino acid sequence selected from the group consisting of SEQ ID NO: 36; the VL CDR1 includes the amino acid sequence of SEQ ID NO: 45; the VL CDR2 includes the amino acid sequence of SEQ ID NO: 50; and the VL CDR3 includes an amino acid sequence selected from the group consisting SEQ ID NO: 55.

In some embodiments, the VH CDR2 is PTM de-risked. In some embodiments, the VH CDR1 includes the amino acid sequence of SEQ ID NO: 22; the VH CDR2 includes the amino acid sequence of SEQ ID NO: 29 or 70; the VH CDR3 includes an amino acid sequence selected from the group consisting of SEQ ID NO: 36; the VL CDR1 includes the amino acid sequence of SEQ ID NO: 45; the VL CDR2 includes the amino acid sequence of SEQ ID NO: 50; and the VL CDR3 includes an amino acid sequence selected from the group consisting SEQ ID NO: 55.

In some embodiments, the VH includes the amino acid sequence of SEQ ID NO: 9 or a sequence having at least 75%, 80%, 85%, 90%, 95% or 99% sequence identity to SEQ ID NO: 9, while retaining the VH CDRs of SEQ ID NO: 9 or PTM re-risked versions thereof. In some embodiments, the VL includes the amino acid sequence of SEQ ID NO: 10 or a sequence having at least 75%, 80%, 85%, 90%, 95% or 99% sequence identity to SEQ ID NO: 10, while retaining the VL CDRs of SEQ ID NO: 10 or PTM re-risked versions thereof. In some embodiments, the VH includes the amino acid sequence of SEQ ID NO: 9, and the VL includes the amino acid sequence of SEQ ID NO: 10.

Also provided, in some embodiments, are antibodies and antigen-binding fragments therefore that bind to the same epitope on CCR8 as 101D5G10G4. Also provided, in some embodiments, are antibodies and antigen-binding fragments therefore that compete with 101D5G10G4 in binding to CCR8.

In some embodiments, provided is an antibody or antigen-binding fragment that is derived from antibody 115C5E3B8. In some embodiments, the VH CDR1 includes the amino acid sequence of SEQ ID NO: 22; the VH CDR2 includes the amino acid sequence of SEQ ID NO: 30; the VH CDR3 includes an amino acid sequence selected from the group consisting of SEQ ID NO: 38; the VL CDR1 includes the amino acid sequence of SEQ ID NO: 44; the VL CDR2 includes the amino acid sequence of SEQ ID NO: 51; and the VL CDR3 includes an amino acid sequence selected from the group consisting SEQ ID NO: 56.

In some embodiments, the VH CDR2 is PTM de-risked. In some embodiments, the VH CDR1 includes the amino acid sequence of SEQ ID NO: 22; the VH CDR2 includes the amino acid sequence of SEQ ID NO: 30 or 71; the VH CDR3 includes an amino acid sequence selected from the group consisting of SEQ ID NO: 38; the VL CDR1 includes the amino acid sequence of SEQ ID NO: 44; the VL CDR2 includes the amino acid sequence of SEQ ID NO: 51; and the VL CDR3 includes an amino acid sequence selected from the group consisting SEQ ID NO: 56.

In some embodiments, the VH includes the amino acid sequence of SEQ ID NO: 11 or a sequence having at least 75%, 80%, 85%, 90%, 95% or 99% sequence identity to SEQ ID NO: 11, while retaining the VH CDRs of SEQ ID NO: 11 or PTM re-risked versions thereof. In some embodiments, the VL includes the amino acid sequence of SEQ ID NO: 12 or a sequence having at least 75%, 80%, 85%, 90%, 95% or 99% sequence identity to SEQ ID NO: 12, while retaining the VL CDRs of SEQ ID NO: 12 or PTM re-risked versions thereof. In some embodiments, the VH includes the amino acid sequence of SEQ ID NO: 11, and the VL includes the amino acid sequence of SEQ ID NO: 12.

Also provided, in some embodiments, are antibodies and antigen-binding fragments therefore that bind to the same epitope on CCR8 as 115C5E3B8. Also provided, in some embodiments, are antibodies and antigen-binding fragments therefore that compete with 115C5E3B8 in binding to CCR8.

In some embodiments, provided is an antibody or antigen-binding fragment that is derived from antibody 163H9D5. In some embodiments, the VH CDR1 includes the amino acid sequence of SEQ ID NO: 23; the VH CDR2 includes the amino acid sequence of SEQ ID NO: 31; the VH CDR3 includes an amino acid sequence selected from the group consisting of SEQ ID NO: 39; the VL CDR1 includes the amino acid sequence of SEQ ID NO: 46; the VL CDR2 includes the amino acid sequence of SEQ ID NO: 52; and the VL CDR3 includes an amino acid sequence selected from the group consisting SEQ ID NO: 57.

In some embodiments, the VL CDR1 is PTM de-risked. In some embodiments, the VH CDR1 includes the amino acid sequence of SEQ ID NO: 23; the VH CDR2 includes the amino acid sequence of SEQ ID NO: 31; the VH CDR3 includes an amino acid sequence selected from the group consisting of SEQ ID NO: 39; the VL CDR1 includes the amino acid sequence of SEQ ID NO: 46, 72 or 73; the VL CDR2 includes the amino acid sequence of SEQ ID NO: 52; and the VL CDR3 includes an amino acid sequence selected from the group consisting SEQ ID NO: 57.

In some embodiments, the VH includes the amino acid sequence of SEQ ID NO: 13 or a sequence having at least 75%, 80%, 85%, 90%, 95% or 99% sequence identity to SEQ ID NO: 13, while retaining the VH CDRs of SEQ ID NO: 13 or PTM re-risked versions thereof. In some embodiments, the VL includes the amino acid sequence of SEQ ID NO: 14 or a sequence having at least 75%, 80%, 85%, 90%, 95% or 99% sequence identity to SEQ ID NO: 14, while retaining the VL CDRs of SEQ ID NO: 14 or PTM re-risked versions thereof. In some embodiments, the VH includes the amino acid sequence of SEQ ID NO: 13, and the VL includes the amino acid sequence of SEQ ID NO: 14.

Also provided, in some embodiments, are antibodies and antigen-binding fragments therefore that bind to the same epitope on CCR8 as 163H9D5. Also provided, in some embodiments, are antibodies and antigen-binding fragments therefore that compete with 163H9D5 in binding to CCR8.

In some embodiments, provided is an antibody or antigen-binding fragment that is derived from antibody 187B5F10. In some embodiments, the VH CDR1 includes the amino acid sequence of SEQ ID NO: 24; the VH CDR2 includes the amino acid sequence of SEQ ID NO: 32; the VH CDR3 includes an amino acid sequence selected from the group consisting of SEQ ID NO: 40; the VL CDR1 includes the amino acid sequence of SEQ ID NO: 47; the VL CDR2 includes the amino acid sequence of SEQ ID NO: 52; and the VL CDR3 includes an amino acid sequence selected from the group consisting SEQ ID NO: 57.

In some embodiments, the VL CDR1 is PTM de-risked. In some embodiments, the VH CDR1 includes the amino acid sequence of SEQ ID NO: 24; the VH CDR2 includes the amino acid sequence of SEQ ID NO: 32, 100, or 101; the VH CDR3 includes an amino acid sequence selected from the group consisting of SEQ ID NO: 40; the VL CDR1 includes the amino acid sequence of SEQ ID NO: 47, 74 or 75; the VL CDR2 includes the amino acid sequence of SEQ ID NO: 52; and the VL CDR3 includes an amino acid sequence selected from the group consisting SEQ ID NO: 57.

In some embodiments, the VH includes the amino acid sequence of SEQ ID NO: 15 or a sequence having at least 75%, 80%, 85%, 90%, 95% or 99% sequence identity to SEQ ID NO: 15, while retaining the VH CDRs of SEQ ID NO: 15 or PTM re-risked versions thereof. In some embodiments, the VL includes the amino acid sequence of SEQ ID NO: 16 or a sequence having at least 75%, 80%, 85%, 90%, 95% or 99% sequence identity to SEQ ID NO: 16, while retaining the VL CDRs of SEQ ID NO: 16 or PTM re-risked versions thereof. In some embodiments, the VH includes the amino acid sequence of SEQ ID NO: 15, and the VL includes the amino acid sequence of SEQ ID NO: 16.

Also provided, in some embodiments, are antibodies and antigen-binding fragments therefore that bind to the same epitope on CCR8 as 187B5F10. Also provided, in some embodiments, are antibodies and antigen-binding fragments therefore that compete with 187B5F10 in binding to CCR8.

In some embodiments, provided is an antibody or antigen-binding fragment that is derived from antibody 195H8D10. In some embodiments, the VH CDR1 includes the amino acid sequence of SEQ ID NO: 24; the VH CDR2 includes the amino acid sequence of SEQ ID NO: 32; the VH CDR3 includes an amino acid sequence selected from the group consisting of SEQ ID NO: 41; the VL CDR1 includes the amino acid sequence of SEQ ID NO: 47; the VL CDR2 includes the amino acid sequence of SEQ ID NO: 52; and the VL CDR3 includes an amino acid sequence selected from the group consisting SEQ ID NO: 57.

In some embodiments, the VL CDR1 is PTM de-risked. In some embodiments, the VH CDR1 includes the amino acid sequence of SEQ ID NO: 24; the VH CDR2 includes the amino acid sequence of SEQ ID NO: 32, 100 or 101; the VH CDR3 includes an amino acid sequence selected from the group consisting of SEQ ID NO: 41; the VL CDR1 includes the amino acid sequence of SEQ ID NO: 47, 74 or 75; the VL CDR2 includes the amino acid sequence of SEQ ID NO: 52; and the VL CDR3 includes an amino acid sequence selected from the group consisting SEQ ID NO: 57.

In some embodiments, the VH includes the amino acid sequence of SEQ ID NO: 17 or a sequence having at least 75%, 80%, 85%, 90%, 95% or 99% sequence identity to SEQ ID NO: 17, while retaining the VH CDRs of SEQ ID NO: 17 or PTM re-risked versions thereof. In some embodiments, the VL includes the amino acid sequence of SEQ ID NO: 18 or a sequence having at least 75%, 80%, 85%, 90%, 95% or 99% sequence identity to SEQ ID NO: 18, while retaining the VL CDRs of SEQ ID NO: 18 or PTM re-risked versions thereof. In some embodiments, the VH includes the amino acid sequence of SEQ ID NO: 17, and the VL includes the amino acid sequence of SEQ ID NO: 18.

Humanized versions of 195H8D10 are also provided. Example humanized VH sequences include SEQ ID NO: 76-79 and example humanized VL sequences include SEQ ID NO: 80-83. In some embodiments, the VH includes the amino acid sequence of SEQ ID NO: 76, 77, 78 or 79 or a sequence having at least 75%, 80%, 85%, 90%, 95% or 99% sequence identity to SEQ ID NO: 76, 77, 78 or 79, while retaining the VH CDRs. In some embodiments, the VL includes the amino acid sequence of SEQ ID NO: 80, 81, 82 or 83 or a sequence having at least 75%, 80%, 85%, 90%, 95% or 99% sequence identity to SEQ ID NO: 80, 81, 82 or 83, while retaining the VL CDRs. In some embodiments, the VH includes the amino acid sequence of SEQ ID NO: 76, 77, 78 or 79, and the VL includes the amino acid sequence of SEQ ID NO: 80, 81, 82 or 83.

PTM re-risked humanized versions of 195H8D10 are also provided. Example humanized VH sequences include SEQ ID NO: 76-79 and example humanized VL sequences include SEQ ID NO: 92-95. In some embodiments, the VH includes the amino acid sequence of SEQ ID NO: 76, 77, 78 or 79 or a sequence having at least 75%, 80%, 85%, 90%, 95% or 99% sequence identity to SEQ ID NO: 76, 77, 78 or 79, while retaining the VH CDRs. In some embodiments, the VL includes the amino acid sequence of SEQ ID NO: 92, 93, 94 or 95 or a sequence having at least 75%, 80%, 85%, 90%, 95% or 99% sequence identity to SEQ ID NO: 92, 93, 94 or 95, while retaining the VL CDRs. In some embodiments, the VH includes the amino acid sequence of SEQ ID NO: 76, 77, 78 or 79, and the VL includes the amino acid sequence of SEQ ID NO: 92, 93, 94 or 95. In some embodiments, the VH includes the amino acid sequence of SEQ ID NO: 76, and the VL includes the amino acid sequence of SEQ ID NO: 92. In some embodiments, the VH includes the amino acid sequence of SEQ ID NO: 77, and the VL includes the amino acid sequence of SEQ ID NO: 94.

Also provided, in some embodiments, are antibodies and antigen-binding fragments therefore that bind to the same epitope on CCR8 as 195H8D10. Also provided, in some embodiments, are antibodies and antigen-binding fragments therefore that compete with 195H8D10 in binding to CCR8.

In some embodiments, provided is an antibody or antigen-binding fragment that is derived from antibody 200C9B9. In some embodiments, the VH CDR1 includes the amino acid sequence of SEQ ID NO: 24; the VH CDR2 includes the amino acid sequence of SEQ ID NO: 33; the VH CDR3 includes an amino acid sequence selected from the group consisting of SEQ ID NO: 42; the VL CDR1 includes the amino acid sequence of SEQ ID NO: 46; the VL CDR2 includes the amino acid sequence of SEQ ID NO: 52; and the VL CDR3 includes an amino acid sequence selected from the group consisting SEQ ID NO: 57.

In some embodiments, the VL CDR1 is PTM de-risked. In some embodiments, the VH CDR1 includes the amino acid sequence of SEQ ID NO: 24; the VH CDR2 includes the amino acid sequence of SEQ ID NO: 33, 102 or 103; the VH CDR3 includes an amino acid sequence selected from the group consisting of SEQ ID NO: 42; the VL CDR1 includes the amino acid sequence of SEQ ID NO: 46, 72 or 73; the VL CDR2 includes the amino acid sequence of SEQ ID NO: 52; and the VL CDR3 includes an amino acid sequence selected from the group consisting SEQ ID NO: 57.

In some embodiments, the VH includes the amino acid sequence of SEQ ID NO: 19 or a sequence having at least 75%, 80%, 85%, 90%, 95% or 99% sequence identity to SEQ ID NO: 19, while retaining the VH CDRs of SEQ ID NO: 19 or PTM re-risked versions thereof. In some embodiments, the VL includes the amino acid sequence of SEQ ID NO: 20 or a sequence having at least 75%, 80%, 85%, 90%, 95% or 99% sequence identity to SEQ ID NO: 20, while retaining the VL CDRs of SEQ ID NO: 20 or PTM re-risked versions thereof. In some embodiments, the VH includes the amino acid sequence of SEQ ID NO: 19, and the VL includes the amino acid sequence of SEQ ID NO: 20.

Humanized versions of 200C9B9 are also provided. Example humanized VH sequences include SEQ ID NO: 84-87 and example humanized VL sequences include SEQ ID NO: 88-91. In some embodiments, the VH includes the amino acid sequence of SEQ ID NO: 84, 85, 86 or 87, or a sequence having at least 75%, 80%, 85%, 90%, 95% or 99% sequence identity to SEQ ID NO: 84, 85, 86 or 87, while retaining the VH CDRs. In some embodiments, the VL includes the amino acid sequence of SEQ ID NO: 88, 89, 90 or 91, or a sequence having at least 75%, 80%, 85%, 90%, 95% or 99% sequence identity to SEQ ID NO: 88, 89, 90 or 91, while retaining the VL CDRs. In some embodiments, the VH includes the amino acid sequence of SEQ ID NO: 84, 85, 86 or 87, and the VL includes the amino acid sequence of SEQ ID NO: 88, 89, 90 or 91.

PTM re-risked humanized versions of 200C9B9 are also provided. Example humanized VH sequences include SEQ ID NO: 84-87 and example humanized VL sequences include SEQ ID NO: 96-99. In some embodiments, the VH includes the amino acid sequence of SEQ ID NO: 84, 85, 86 or 87, or a sequence having at least 75%, 80%, 85%, 90%, 95% or 99% sequence identity to SEQ ID NO: 84, 85, 86 or 87, while retaining the VH CDRs. In some embodiments, the VL includes the amino acid sequence of SEQ ID NO: 96, 97, 98 of 99, or a sequence having at least 75%, 80%, 85%, 90%, 95% or 99% sequence identity to SEQ ID NO: 96, 97, 98 of 99, while retaining the VL CDRs. In some embodiments, the VH includes the amino acid sequence of SEQ ID NO: 84, 85, 86 or 87, and the VL includes the amino acid sequence of SEQ ID NO: 96, 97, 98 of 99. In some embodiments, the VH includes the amino acid sequence of SEQ ID NO: 85, and the VL includes the amino acid sequence of SEQ ID NO: 98. In some embodiments, the VH includes the amino acid sequence of SEQ ID NO: 84, and the VL includes the amino acid sequence of SEQ ID NO: 98.

Also provided, in some embodiments, are antibodies and antigen-binding fragments therefore that bind to the same epitope on CCR8 as 200C9B9. Also provided, in some embodiments, are antibodies and antigen-binding fragments therefore that compete with 200C9B9 in binding to CCR8.

Also provided, in some embodiments, are antibodies and antigen-binding fragments that include CDR sequences derived from the presently disclosed CDR sequences, with one, two or three amino acid substitutions, deletions, and/or additions.

In some embodiments, the anti-CCR8 antibodies are modified mAbs comprising a modified heavy chain constant region, such as an afucosylated heavy chain, that binds with higher affinity to activating Fcγ receptor that mediated enhanced ADCC compared to an unmodified mAb. In some embodiments, the anti-CCR8 antibodies comprises a heavy chain which is of a human IgG1 variant that include the single or combination of L234Y, L235Q, G236W, S239D/M, F243L, H268D, D270E, R292P, S298A, Y300L, V305I, K326D, A330L/M, 1332E, K334A/E, P396L that enhance ADCC function (all EU numbering).

In some embodiments, the human IgG1 Fc is a Fc-DLE (S239D/A330L/1332E EU numbering), which is preferably symmetric. In some embodiments, the human IgG1 Fc is a Fc-DE (S239D/1332E EU numbering), which is preferably symmetric. In some embodiments, the human IgG1 is afucosylated. In some embodiments, the human IgG1 Fc is asymmetric. For instance, one of the Fc chains includes one or more (or all) of substitutions L234Y/L235Q/G236W/S239M/H268D/D270E/S298A and opposing Fc chain includes one or more (or all) of D270E/K326D/A330M/K334E (EU numbering). In certain preferred embodiments, the IgG1 Fc is Fc-DLE, afucosylated or asymmetric.

### Multi-Functional Molecules

Multi-functional molecules that include an antibody or antigen-binding fragment specific to CCR8, such as those disclosed herein, and one or more antibody or antigen-binding fragment having specificity to a second antigen.

In some embodiments, the second antigen is a protein expressed on an immune cell, such as a T cell, a B cell, a monocyte, a macrophage, a neutrophil, a dendritic cell, a phagocyte, a natural killer cell, an eosinophil, a basophil, and a mast cell.

In some embodiments, the second antigen is CD3, CD47, PD1, PD-L1, LAG3, TIM3, CTLA4, VISTA, CSFR1, A2AR, CD73, CD39, CD40, CEA, HER2, CMET, 4-1BB, OX40, SIRPA, CD28, ICOS, CTLA4, BTLA, TIGIT, HVEM, CD27, VEGFR, or VEGF.

Different formats of bispecific antibodies are also provided. In some embodiments, each of the anti-CCR8 fragment and the second fragment each is independently selected from a Fab fragment, a single-chain variable fragment (scFv), or a single-domain antibody. In some embodiments, the bispecific antibody further includes a Fc fragment.

Bifunctional molecules that include not just antibody or antigen binding fragment are also provided. As a tumor antigen targeting molecule, an antibody or antigen-binding fragment specific to CCR8, such as those described here, can be combined with an immune cytokine or ligand optionally through a peptide linker. The linked immune cytokines or ligands include, but not limited to, IL-2, IL-3, IL-4, IL-5, IL-6, IL-7, IL-10, IL-12, IL-13, IL-15, GM-CSF, TNF-α, CD40L, OX40L, CD27L, CD30L, 4-1BBL, LIGHT and GITRL. Such bi-functional molecules can combine the immune checkpoint blocking effect with tumor site local immune modulation.

### Chimeric Antigen Receptors

Also provided, in one embodiment, is a chimeric antigen receptor (CAR) that includes the antibody or fragment thereof of the present disclosure as a targeting unit. In some embodiments, the CAR includes an antibody or fragment thereof of the present disclosure, a transmembrane domain, a costimulatory domain, and a CD38 intracellular domain.

A transmembrane domain can be designed to be fused to the extracellular domain which includes the antibody or fragment, optionally through a hinge domain. It can similarly be fused to an intracellular domain, such as a costimulatory domain. In some embodiments, the transmembrane domain can include the natural transmembrane region of a costimulatory domain (e.g., the TM region of a CD28 or 4-1BB employed as a costimulatory domain) or the natural transmembrane domain of a hinge region (e.g., the TM region of a CD8 alpha or CD28 employed as a hinge domain).

In some embodiments, the transmembrane domain can include a sequence that spans a cell membrane, but extends into the cytoplasm of a cell and/or into the extracellular space.

For example, a transmembrane can include a membrane-spanning sequence which itself can further include 1, 2, 3, 4, 5, 6, 7, 8, 9, 10 or more amino acids that extend into the cytoplasm of a cell, and/or the extracellular space. Thus, a transmembrane domain includes a membrane-spanning region, yet can further comprise an amino acid(s) that extend beyond the internal or external surface of the membrane itself; such sequences can still be considered to be a “transmembrane domain”.

In some embodiments, the transmembrane domain is fused to the cytoplasmic domain through a short linker. Optionally, the short peptide or polypeptide linker, preferably between 2 and 10 amino acids in length can form the linkage between the transmembrane domain and a proximal cytoplasmic signaling domain of the chimeric receptor. A glycine-serine doublet (GS), glycine-serine-glycine triplet (GSG), or alanine-alanine-alanine triplet (AAA) provides a suitable linker.

In some embodiments, the CAR further includes a costimulatory domain. In some embodiments, the costimulatory domain is positioned between the transmembrane domain and an activating domain. Example costimulatory domains include, but are not limited to, CD2, CD3 delta, CD3 epsilon, CD3 gamma, CD4, CD7, CD8a, CD8, CD11a (ITGAL), CD11b (ITGAM), CD11c (ITGAX), CD11d (ITGAD), CD18 (ITGB2), CD19 (B4), CD27 (T FRSF7), CD28, CD28T, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A (B-cell antigen receptor complex-associated alpha chain), CD79B (B-cell antigen receptor complex-associated beta chain), CD84 (SLAMF5), CD96 (Tactile), CD 100 (SEMA4D), CD 103 (ITGAE), CD134 (OX40), CD137 (4-1BB), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KTR3DL2), CD160 (BY55), CD162 (SELPLG), CD226 (DNAMI), CD229 (SLAMF3), CD244 (SLAMF4), CD247 (CD3-zeta), CD258 (LIGHT), CD268 (BAFFR), CD270 (T FSF14), CD272 (BTLA), CD276 (B7-H3), CD279 (PD-1), CD314 (KG2D), CD319 (SLAMF7), CD335 (K-p46), CD336 (K-p44), CD337 (K-p30), CD352 (SLAMF6), CD353 (SLAMF8), CD355 (CRTAM), CD357 (TNFRSF 18), inducible T cell co-stimulator (ICOS), LFA-1 (CD 1 1a/CD 18), KG2C, DAP-10, ICAM-1, Kp80 (KLRF1), IL-2R beta, IL-2R gamma, IL-7R alpha, LFA-1, SLAMF9, LAT, GADS (GrpL), SLP-76 (LCP2), PAG1/CBP, a CD83 ligand, Fc gamma receptor, MHC class 1 molecule, MHC class 2 molecule, a TNF receptor protein, an immunoglobulin protein, a cytokine receptor, an integrin, activating NK cell receptors, a Toll ligand receptor, and fragments or combinations thereof.

In some embodiments, the cytoplasmic portion of the CAR also includes a signaling/activation domain. In one embodiment, the signaling/activation domain is the CD38 domain, or is an amino acid sequence having at least about 80%, 85%, 90%, 95%, 98% or 99% sequence identity to the CD38 domain.

Polynucleotides, mRNA, and Methods of Expressing or Preparing Antibodies

The present disclosure also provides polynucleotides or nucleic acid molecules encoding the antibodies, variants or derivatives thereof of the disclosure, or the CAR. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

In some embodiments, the polynucleotide is an mRNA molecule. In some embodiments, the mRNA can be introduced into a target cell for expressing the antibody or fragment thereof.

mRNAs may be synthesized according to any of a variety of known methods. For example, the mRNAs may be synthesized via in vitro transcription (IVT). Briefly, IVT is typically performed with a linear or circular DNA template containing a promoter, a pool of ribonucleotide triphosphates, a buffer system that may include DTT and magnesium ions, and an appropriate RNA polymerase (e.g., T3, T7 or SP6 RNA polymerase), DNAse I, pyrophosphatase, and/or RNAse inhibitor. The exact conditions will vary according to the specific application.

In some embodiments, for the preparation of antibody-coding mRNA, a DNA template is transcribed in vitro. A suitable DNA template typically has a promoter, for example a T3, T7 or SP6 promoter, for in vitro transcription, followed by desired nucleotide sequence for desired antibody encoding (e.g., heavy chain or light chain encoding) mRNA and a termination signal.

Desired antibody encoding (e.g., heavy chain or light chain encoding) mRNA sequence may be determined and incorporated into a DNA template using standard methods. For example, starting from a desired amino acid sequence (e.g., a desired heavy chain or light chain sequence), a virtual reverse translation is carried out based on the degenerated genetic code. Optimization algorithms may then be used for selection of suitable codons. Typically, the G/C content can be optimized to achieve the highest possible G/C content on one hand, taking into the best possible account the frequency of the tRNAs according to codon usage on the other hand. The optimized RNA sequence can be established and displayed, for example, with the aid of an appropriate display device and compared with the original (wild-type) sequence. A secondary structure can also be analyzed to calculate stabilizing and destabilizing properties or, respectively, regions of the RNA.

The mRNA may be synthesized as unmodified or modified mRNA. Typically, mRNAs are modified to enhance stability. Modifications of mRNA can include, for example, modifications of the nucleotides of the RNA. A modified mRNA can thus include, for example, backbone modifications, sugar modifications or base modifications. In some embodiments, antibody encoding mRNAs (e.g., heavy chain and light chain encoding mRNAs) may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, 13-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosure of which is included here in its full scope by reference.

In some embodiments, the mRNAs (e.g., heavy chain and light chain encoding mRNAs) may contain RNA backbone modifications. Typically, a backbone modification is a modification in which the phosphates of the backbone of the nucleotides contained in the RNA are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, the mRNAs (e.g., heavy chain and light chain encoding mRNAs) may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyloligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, the mRNAs (e.g., heavy chain and light chain encoding mRNAs) may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

Typically, mRNA synthesis includes the addition of a “cap” on the N-terminal (5′) end, and a “tail” on the C-terminal (3′) end. The presence of the cap is important in providing resistance to nucleases found in most eukaryotic cells. The presence of a “tail” serves to protect the mRNA from exonuclease degradation.

Thus, in some embodiments, the mRNAs (e.g., heavy chain and light chain encoding mRNAs) include a 5′ cap structure. A 5′ cap is typically added as follows: first, an RNA terminal phosphatase removes one of the terminal phosphate groups from the 5′ nucleotide, leaving two terminal phosphates; guanosine triphosphate (GTP) is then added to the terminal phosphates via a guanylyl transferase, producing a 5′5′5 triphosphate linkage; and the 7-nitrogen of guanine is then methylated by a methyltransferase. Examples of cap structures include, but are not limited to, m7G(5′)ppp (5′(A,G(5′)ppp(5) A and G(5)ppp(5′)G.

In some embodiments, the mRNAs (e.g., heavy chain and light chain encoding mRNAs) include a 3′ poly(A) tail structure. A poly-A tail on the 3′ terminus of mRNA typically includes about 10 to 300 adenosine nucleotides (e.g., about 10 to 200 adenosine nucleotides, about 10 to 175 adenosine nucleotides, about 10 to 150 adenosine nucleotides, about 10 to 125 adenosine nucleotides, 10 to 100 adenosine nucleotides, about 10 to 75 adenosine nucleotides, about 20 to 70 adenosine nucleotides, or about 20 to 60 adenosine nucleotides). In some embodiments, antibody encoding mRNAs (e.g., heavy chain and light chain encoding mRNAs) include a 3′ poly(C) tail structure. A suitable poly-C tail on the 3′ terminus of mRNA typically include about 10 to 200 cytosine nucleotides (e.g., about 10 to 150 cytosine nucleotides, about 10 to 100 cytosine nucleotides, about 20 to 70 cytosine nucleotides, about 20 to 60 cytosine nucleotides, or about 10 to 40 cytosine nucleotides). The poly-C tail may be added to the poly-A tail or may substitute the poly-A tail.

In some embodiments, the mRNAs (e.g., heavy chain and light chain encoding mRNAs) include a 5′ and/or 3′ untranslated region. In some embodiments, a 5′ untranslated region includes one or more elements that affect an mRNA's stability or translation, for example, an iron responsive element. In some embodiments, a 5′ untranslated region may be between about 50 and 500 nucleotides in length (e.g., about 50 and 400 nucleotides in length, about 50 and 300 nucleotides in length, about 50 and 200 nucleotides in length, or about 50 and 100 nucleotides in length).

In some embodiments, a 5′ region of an mRNA (e.g., heavy chain and light chain encoding mRNAs) includes a sequence encoding a signal peptide, such as those described herein. In particular embodiments, a signal peptide derived from human growth hormone (hGH) is incorporated in the 5′ region. Typically, a signal peptide encoding sequence is linked, directly or indirectly, to the heavy chain or light chain encoding sequence at the N-terminus.

The present technology may be used to deliver any antibody known in the art and antibodies that can be produced against desired antigens using standard methods. The present invention may be used to deliver monoclonal antibodies, polyclonal antibodies, antibody mixtures or cocktails, human or humanized antibodies, chimeric antibodies, or bi-specific antibodies.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

### Treatment and Uses

As described herein, the antibodies, variants, derivatives or antibody-drug conjugates of the present disclosure may be used in certain treatment and diagnostic methods.

The present disclosure is further directed to antibody-based therapies which involve administering the antibodies, fragments, or antibody-drug conjugates of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. As provided above, CCR8 can be overexpressed in tumor cells, in particular liver, gastric, pancreatic, esophageal, ovarian, and lung tumors. Inhibition of CCR8 has been shown to be useful for treating the tumors.

Accordingly, in some embodiments, provided are methods for treating a cancer in a patient in need thereof. The method, in one embodiment, entails administering to the patient an effective amount of an antibody, fragment, or antibody-drug conjugate of the present disclosure. In some embodiments, at least one of the cancer cells (e.g., stromal cells) in the patient over-express CCR8.

Cellular therapies, such as chimeric antigen receptor (CAR) T-cell therapies, are also provided in the present disclosure. A suitable cell can be used, that is transduced with a vector that encodes, or put in contact with, an CAR that includes an anti-CCR8 antibody of the present disclosure (or alternatively engineered to express an anti-CCR8 antibody of the present disclosure). Upon such contact or engineering, the cell can then be introduced to a cancer patient in need of a treatment. The cancer patient may have a cancer of any of the types as disclosed herein. The cell (e.g., T cell) can be, for instance, a tumor-infiltrating T lymphocyte, a CD4+ T cell, a CD8+ T cell, or the combination thereof, without limitation.

In some embodiments, the cell was isolated from the cancer patient him- or her-self. In some embodiments, the cell was provided by a donor or from a cell bank. When the cell is isolated from the cancer patient, undesired immune reactions can be minimized.

Non-limiting examples of cancers include bladder cancer, breast cancer, colorectal cancer, endometrial cancer, esophageal cancer, head and neck cancer, kidney cancer, leukemia, liver cancer, lung cancer, lymphoma, melanoma, pancreatic cancer, prostate cancer, and thyroid cancer. In some embodiments, the cancer is one or more of gastric, pancreatic, esophageal, ovarian, lung cancers and cutaneous T cell lymphoma.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

CCR8 is expressed on monocytes and Th2 lymphocytes and in the brain, spleen and thymus. It is the receptor for the chemokine CCL1, which is chemotactic for Th2 cells. CCL1 has also shown to be involved in eosinophil recruitment. The antibodies disclosed herein can be used to inhibit CCR8 activity; to inhibit CCL1 activity and to inhibit or treat (therapeutically or prophylactically) conditions mediated by CCR8 and/or CCL1, including, but not limited to inflammatory disorders and allergic conditions. The disclosed antibodies can also be advantageously used to inhibit conditions mediated by eosinophils and by monocytes, T lymphocytes and other immune system cells that express CCR8, including inflammatory disorders and allergic conditions mediated by these cells (e.g., asthma, atopic dermatitis, and allergic rhinitis), as well as T cell leukemia and lymphoma.

In addition, CCR8 acts as a co-receptor for HIV infection, along with CD4, for some HIV-1 strains when transfected into cells that did not express either of the major co-receptors CCR5 and CXCR4. Thus, in one embodiment of the invention, a CCR8 monoclonal antibody is used to block the interaction of CCR8 with HIV surface proteins and thereby prevent HIV infection of the CCR8-expressing cell.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibody, fragment, or antibody-drug conjugate or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracisternally, intravaginally, intraperitoneally, topically (as by powders, ointments, drops or transdermal patch), buccally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antigen-binding polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

The amount of the antibodies, fragments, or antibody-drug conjugates of the disclosure which will be effective in the treatment, inhibition and prevention of an inflammatory, immune or malignant disease, disorder or condition can be determined by standard clinical techniques. In addition, in vitro assays may optionally be employed to help identify optimal dosage ranges. The precise dose to be employed in the formulation will also depend on the route of administration, and the seriousness of the disease, disorder or condition, and should be decided according to the judgment of the practitioner and each patient's circumstances. Effective doses may be extrapolated from dose-response curves derived from in vitro or animal model test systems.

As a general proposition, the dosage administered to a patient of the antibodies, fragments, or antibody-drug conjugates of the present disclosure is typically 0.001 mg/kg to 100 mg/kg of the patient's body weight, between 0.01 mg/kg and 20 mg/kg of the patient's body weight, or 0.5 mg/kg to 10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the disclosure may be reduced by enhancing uptake and tissue penetration (e.g., into the brain) of the antibodies by modifications such as, for example, lipidation.

In an additional embodiment, the compositions of the disclosure are administered in combination with cytokines. Cytokines that may be administered with the compositions of the disclosure include, but are not limited to, IL-2, IL-3, IL-4, IL-5, IL-6, IL-7, IL-10, IL-12, IL-13, IL-15, anti-CD40, CD40L, and TNF-α.

In additional embodiments, the compositions of the disclosure are administered in combination with other therapeutic or prophylactic regimens, such as, for example, radiation therapy.

### Compositions

The present disclosure also provides pharmaceutical compositions. Such compositions comprise an effective amount of an antibody, fragment, or antibody-drug conjugate, and an acceptable carrier. In some embodiments, the composition further includes a second anticancer agent (e.g., an immune checkpoint inhibitor).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1. Generation and Testing of Mouse Anti Human CCR8 Antibodies

This example describes the generation of mouse anti-human CCR8 monoclonal antibodies using the hybridoma technology.

Antigen: Plasmid DNA encoding human CCR8 or CHO-K1 cell line that expressed human CCR8 (CHO-K1 hCCR8 cell). Plasmid DNA immunization was performed by hydrodynamic tail vein injection. Cellular immunization was performed by intraperitoneal (i.p.) injection of CHO-K1 hCCR8 cell line.

Immunization: To generate monoclonal antibodies against human CCR8, Balb/c mice, C57BL/6 mice and SJL mice, were immunized with the full length CCR8 DNA or CHO-K1 hCCR8 cell line. To monitor immune responses, titrated serum from mice was screened by flow cytometry as described below, typically 4-6 weeks of immunizations. Serum was screened for antibody binding to multiple CCR8 cell lines, and the corresponding negative control cell lines not expressing CCR8. CCR8 specific and non-specific Ab responses were measured in each animal, and animals with enough titers of anti-hCCR8 Ig were selected for final boost with HEK293 hCCR8 cell line 3-6 days before hybridoma fusions.

Cell fusion and Hybridoma screening: The spleens were isolated from final boosted mice as described above. Hybridomas were generated by cell fusions with immortalized mouse myeloma cells by electric field-based electrofusion. The fused cells were plated in 96 flat-bottom microtiter plates for selection of hybridomas. The supernatants were screened by FACS using HEK293 hCCR8 cell line, CHO-K1 cyno CCR8 cell line and the negative control cell line.

Subcloning and screening: Positive primary clones from each fusion were subcloned by limiting dilutions to ensure that the subclones were derived from a single parental cell. Subcloning were screened in the same approach as primary clones and additionally, to identify the blocking antibodies, culture supernatant of positive clones underwent another round of confirmative screening by signaling assay using β-arrestin hCCR8 CHO-K1 cell line.

Hybridoma clones 84D1-2H3,86D4E12A5,96G3-1F10,99D1-1E11,101D5G10G4, 115C5E3B8,163H9D5,187B5F10,195H8D10 and 200C9B9 were selected for further analysis. The amino acid sequences of the variable regions are provided in Table 1 below, with the CDR sequences summarized in Table 1A.

TABLE 1
Antibody Variable Region Sequences

| Name | Sequence | SEQ ID NO: |
|---|---|---|
| 84D1-2H3 VH | EVQLQQSGPVLVKPGASVKMSCKASGYTFTDYYVNWVKKSHGKSPE | 1 |
| | WIGIIKPYNGGTSYNQKFKGKATLTVDKSSSTVYMELNSLTSEDSA | |
| | VYHCARFAYSRNPYAMDYWGQGTSVTVSS | |
| 84D1-2H3 VL | DVLLTQTPLSLPVSLGDQASISCRSSQSIVHSTGNTYLEWYLQKPG | 2 |
| | QSPKVLIYKVSTRFSGVPDRFSGSGSGTDFTLKISRVEAEDLGVYY | |
| | CFQGSHVPPTFGGGTKLEIK | |
| 86D4E12A5 VH | DLQLKESGPGLVKPSQSLSLTCSVTGYSITSGYYWNWIRQLPGNKL | 3 |
| | EWMGYISFDGSNNYNPSLKTRISITRDTSKNQFFLKLNSVTSEDTA | |
| | TYFCARLRGLLLGHFDVWGTGTTVTVSS | |
| 86D4E12A5 VL | DVQMTQSSSYLSVPLGGRVTITCKASDHINNRLAWYQQKPGNAPRL | 4 |
| | LISGATKLETGIPSRFRGSGSGKDYTLSIFSLQTEDVATYYCQQYW | |
| | STSYTFGGGTKLELR | |
| 96G3-1F10 VH | DLQLKESGPGLVKPSQSLSLTCSVTGYSITSGYYWNWIRQFPGNKL | 5 |
| | EWMGFISYDGNNNYNPSLKNRISITRDTSKNQFFLNVKSVTTEDTA | |
| | TYYCSRATGLRKAWFAYWGQGTLVTVSA | |
| 96G3-1F10 VL | DIQMTQSSSYLSVSLGGRVTITCKASDHINNWLAWYQQKPGNAPRL | 6 |
| | LISDTTSLGTGVPSRFRGSGSGKDYTLSITSLQTEDVATYFCQQYW | |
| | SYPYTFGGGTKLEIR | |
| 99D1-1E11 VH | DLQLKESGPGLVKPSQSLSLTCSVTGYSITSGYYWNWIRQFPGNKL | 7 |
| | EWMGYISYDGSNNYNPSLKTRISITRDTSKNQFFLKLNSVTSEDTA | |
| | TYYCVRERGLLLGHFDVWGTGTTVTVSS | |
| 99D1-1E11 VL | DIQMTQSSSYLSVALGGRVTITCKASDHINNRLAWYQQKPGNAPRL | 8 |
| | LISGATKLETGIPSRFRGSGSGKDYTLSIFSLQTEDVATYYCQQYW | |
| | STSYTFGGGTKLELR | |
| 101D5G10G4 VH | DLQLKESGPGLVKPSQSLSLTCSVTGYSITSGYYWNWIRQFPGNKL | 9 |
| | EWMGYISYDGNNNYNPSLKNRISITRDTSKNQFFLNLNSVTTEDTA | |
| | TYYCSRATGLRKAWFAYWGQGTLVTVSA | |
| 101D5G10G4 VL | DIQMTQSSSYLSVSLGGRVTITCKASDHINNWLAWYQQKPGNAPRL | 10 |
| | LISDTTSLGTGVPSRFRGSGSGKDYTLSITSLQTEDVATYFCQQYW | |
| | SYPYTFGGGTKLEIR | |
| 115C5E3B8 VH | DVQLQESGPGLVKPSQSLSLTCSVTGYSITSGYYWNWIRQFPGNKL | 11 |
| | EWMGYISYDGYNDYNPSLKNRISITRDTSKNQFFLKLNSVTTEDTA | |
| | TYYCARETGLRKAWFAYWGQGTLVTVSG | |
| 115C5E3B8 VL | DIQMTQSSSYLSVSLGGRVTITCKASDHINNRLAWYQQKPGNAPRL | 12 |
| | LISDTSSLETGVPSRFSGSGSGKDYTLRITSLQTEDVATYYCQQYW | |
| | GTPYTFGGGTKLEIK | |
| 163H9D5 VH | EVQLVESGGGLVQPKGSLKLSCAASGFSFNTYAMNWVRQAPGKGLK | 13 |
| | WVARIRTKSNNYATHYADSVKDRFTLSRDDSESMLYLQMNNLKTED | |
| | TAMYYCVGGGYGSSASNAMAYWGQGTSVTVSS | |
| 163H9D5 VL | DIVMTQAAPSVPVTPGESVSISCRSSKSLLHSNGNTYLYWFLQRPG | 14 |
| | QSPQLLIFRMSNLASGVPDRFSGSGSGTAFTLRISRVEAEDVGVYY | |
| | CMQHLEYPFTFGSGTKLEIK | |
| 187B5F10 VH | EVQLVGSGGGLVQPKGSLKLSCAASGFNFNTYVMNWVRQAPGKGLE | 15 |
| | WVARIRTKSNNYATYYADSVKDRFTISRDDSESKLYLQMNNLKTED | |
| | TAMYYCVSGTITRLGGGMDYWGQGTSVTVSS | |
| 187B5F10 VL | DIVMTQAAPSVPVTPGESVSISCRSSKRLLHSNGNTYLYWFLQRPG | 16 |
| | QSPQLLIYRMSNLASGVPDRFSGSGSGTAFTLRISRVEAEDVGVYY | |
| | CMQHLEYPFTFGGGTKLEIK | |
| 195H8D10 VH | EVQLVGSGGGLVQPKGSLKLSCAASGFNFNTYVMNWVRQAPGKGLE | 17 |
| | WVARIRTKSNNYATYYADSVKDRFTISRDDSESKLYLQMNNLKTED | |
| | TAMYYCVSGTITRLGGGLDYWGQGTSVTVSS | |
| 195H8D10 VL | DIVMTQAAPSVPVTPGESVSISCRSSKRLLHSNGNTYLYWFLQRPG | 18 |
| | QSPQLLIYRMSNLASGVPDRFSGSGSGTAFTLRISRVEAEDVGVYY | |
| | CMQHLEYPFTFGGGTKLEIK | |
| 200C9B9 VH | EVQLVESGGGLVQPKGSLKLSCAASGFSFNTYVMNWVRQAPGKGLN | 19 |
| | WVARIRTKSNNYATFYVDSVKDRFTISRDDSENMLYLQMNNLKIED | |
| | TAIYYCVSGTITRLGAGLDYWGQGTSVTVSS | |
| 200C9B9 VL | DIVMTQAAPSVPVTPGESVSISCRSSKSLLHSNGNTYLYWFLQRPG | 20 |
| | QSPQLLIYRMSNLASGVPDRFSGSGSGTAFTLRISRVEAEDVGVYY | |
| | CMQHLEYPFTFGGGTKLEIK | |

TABLE 1A
CDR Sequences (with optional mutations to avoid PTM, Kabat numbering)

| | VH | SEQ ID | | SEQ ID | | SEQ ID |
|---|---|---|---|---|---|---|
| Antibody | CDR1 | NO: | VH CDR2 | NO: | VH CDR3 | NO: |
| 84D1-2H3 | DYYVN | 21 | IIKPYNGGTSYNQKFKG | 25 | FAYSRNPYAMDY | 34 |
| | | | IIKPYNAGTSYNQKFKG | 65 | | |
| | | | IIKPYQGGTSYNQKFKG | 66 | | |
| 86D4E12A5 | SGYYWN | 22 | YISFDGSNNYNPSLKT | 26 | LRGLLLGHFDV | 35 |
| | | | YISFDASNNYNPSLKT | 67 | | |
| 96G3-1F10 | SGYYWN | 22 | FISYDGNNNYNPSLKN | 27 | ATGLRKAWFAY | 36 |
| | | | FISYDANNNYNPSLKN | 68 | | |
| 99D1-1E11 | SGYYWN | 22 | YISYDGSNNYNPSLKT | 28 | ERGLLLGHFDV | 37 |
| | | | YISYDASNNYNPSLKT | 69 | | |
| 101D5G10G4 | SGYYWN | 22 | YISYDGNNNYNPSLKN | 29 | ATGLRKAWFAY | 36 |
| | | | YISYDANNNYNPSLKN | 70 | | |
| 115C5E3B8 | SGYYWN | 22 | YISYDGYNDYNPSLKN | 30 | ETGLRKAWFAY | 38 |
| | | | YISYDAYNDYNPSLKN | 71 | | |
| 163H9D5 | TYAMN | 23 | RIRTKSNNYATHYADSVKD | 31 | GGYGSSASNAMAY | 39 |
| 187B5F10 | TYVMN | 24 | RIRTKSNNYATYYADSVKD | 32 | GTITRLGGGMDY | 40 |
| | | | RIRTKSNNYATYYADAVKD | 100 | | |
| | | | RIRTKSNNYATYYAESVKD | 101 | | |
| 195H8D10 | TYVMN | 24 | RIRTKSNNYATYYADSVKD | 32 | GTITRLGGGLDY | 41 |
| | | | RIRTKSNNYATYYADAVKD | 100 | | |
| | | | RIRTKSNNYATYYAESVKD | 101 | | |
| 200C9B9 | TYVMN | 24 | RIRTKSNNYATFYVDSVKD | 33 | GTITRLGAGLDY | 42 |
| | | | RIRTKSNNYATFYVDAVKD | 102 | | |
| | | | RIRTKSNNYATFYVESVKD | 103 | | |

Bold/underlined residues indicate mutations to avoid PTM

| | | SEQ ID | | SEQ ID | | SEQ ID |
|---|---|---|---|---|---|---|
| Antibody | VL CDR1 | NO: | VL CDR2 | NO: | VL CDR3 | NO: |
| 84D1-2H3 | RSSQSIVHSTGNTYLE | 43 | KVSTRFS | 48 | FQGSHVPPT | 53 |
| 86D4E12A5 | KASDHINNRLA | 44 | GATKLET | 49 | QQYWSTSYT | 54 |
| 96G3-1F10 | KASDHINNWLA | 45 | DTTSLGT | 50 | QQYWSYPYT | 55 |
| 99D1-1E11 | KASDHINNRLA | 44 | GATKLET | 49 | QQYWSTSYT | 54 |
| 101D5G10G4 | KASDHINNWLA | 45 | DTTSLGT | 50 | QQYWSYPYT | 55 |
| 115C5E3B8 | KASDHINNRLA | 44 | DTSSLET | 51 | QQYWGTPYT | 56 |
| 163H9D5 | RSSKSLLHSNGNTYLY | 46 | RMSNLAS | 52 | MQHLEYPFT | 57 |
| | RSSKSLLHSNANTYLY | 72 | | | | |
| | RSSKSLLHSQGNTYLY | 73 | | | | |
| 187B5F10 | RSSKRLLHSNGNTYLY | 47 | RMSNLAS | 52 | MQHLEYPFT | 57 |
| | RSSKRLLHSNANTYLY | 74 | | | | |
| | RSSKRLLHSQGNTYLY | 75 | | | | |
| 195H8D10 | RSSKRLLHSNGNTYLY | 47 | RMSNLAS | 52 | MQHLEYPFT | 57 |
| | RSSKRLLHSNANTYLY | 74 | | | | |
| | RSSKRLLHSQGNTYLY | 75 | | | | |
| 200C9B9 | RSSKSLLHSNGNTYLY | 46 | RMSNLAS | 52 | MQHLEYPFT | 57 |
| | RSSKSLLHSNANTYLY | 72 | | | | |
| | RSSKSLLHSQGNTYLY | 73 | | | | |

Review of the sequences revealed that antibodies 86D4E12A5, 96G3-1F10, 99D1-1E11, 101D5G10G4, and 115C5E3B8 are homologous between one another, and 163H9D5, 187B5F10, 195H8D10 and 200C9B9 are homologous between one another as well. They are referred to as Group A and Group B, respectively, sharing the following consensus sequences for their CDRs.

TABLE 1B
Consensus CDR sequences of antibody groups

| Group A | Consensus (examples) | Definitions | SEQ ID NO: |
|---|---|---|---|
| VH CDR1 | SGYYWN | | 22 |
| VH CDR2 | X₁ISX₂DX₃X₄NX₅YNPSLKX₆ | X₁ is F or Y, X₂ is F or Y, X₃ is | 58 |
| | (YISFDGSNNYNPSLKT | G or A, X₄ is S, N or Y, X₅ is | 26 |
| | YISFDASNNYNPSLKT | D or N, X₆ is N or T | 67 |
| | FISYDGNNNYNPSLKN | | 27 |
| | FISYDANNNYNPSLKN | | 68 |
| | YISYDGSNNYNPSLKT | | 28 |
| | YISYDASNNYNPSLKT | | 69 |
| | YISYDGNNNYNPSLKN | | 29 |
| | YISYDANNNYNPSLKN | | 70 |
| | YISYDGYNDYNPSLKN | | 30 |
| | YISYDAYNDYNPSLKN) | | 71 |
| VH CDR3 | (LRGLLLGHFDV | | 35 |
| | ATGLRKAWFAY | | 36 |
| | ERGLLLGHFDV | | 37 |
| | ETGLRKAWFAY) | | 38 |
| VL CDR1 | KASDHINNXLA | X is R or W | 59 |
| | (KASDHINNRLA | | 44 |
| | KASDHINNWLA) | | 45 |
| VL CDR2 | (GATKLET | | 49 |
| | DTTSLGT | | 50 |
| | DTSSLET) | | 51 |
| VL CDR3 | QQYWX₁X₂X₃YT | X₁ is G or S, X₂ is T or Y, X₃ | 60 |
| | (QQYWSTSYT | is P or S | 54 |
| | QQYWSYPYT | | 55 |
| | QQYWGTPYT) | | 56 |
| Group B | Consensus | Definition | SEQ ID NO: |
| VH CDR1 | TYXMN | X is A or V | 61 |
| | (TYAMN | | 23 |
| | TYVMN) | | 24 |
| VH CDR2 | RIRTKSNNYATX₁YX₂X₃X₄VKD | X₁ is F, H or Y, X₂ is A or V, | 62 |
| | (RIRTKSNNYATHYADSVKD | X₃X₄ is DS, DA or ES | 31 |
| | RIRTKSNNYATYYADSVKD | | 32 |
| | RIRTKSNNYATYYADAVKD | | 100 |
| | RIRTKSNNYATYYAESVKD | | 101 |
| | RIRTKSNNYATFYVDSVKD | | 33 |
| | RIRTKSNNYATFYVDAVKD | | 102 |
| | RIRTKSNNYATFYVESVKD) | | 103 |
| VH CDR3 | GTITRLGX₁GX₂DY | X₁ is A or G, X₂ is L or M | 63 |
| | (GTITRLGGGMDY | | 40 |
| | GTITRLGGGLDY | | 41 |
| | GTITRLGAGLDY) or | | 42 |
| | GGYGSSASNAMAY | | 39 |
| VL CDR1 | RSSKX₁LLHSX₂X₃NTYLY | X₁ is R or S, X₂ is N or Q, X₃ | 64 |
| | (RSSKSLLHSNGNTYLY | is G or A | 46 |
| | RSSKSLLHSNANTYLY | | 72 |
| | RSSKSLLHSQGNTYLY | | 73 |
| | RSSKRLLHSNGNTYLY | | 47 |
| | RSSKRLLHSNANTYLY | | 74 |
| | RSSKRLLHSQGNTYLY) | | 75 |
| VL CDR2 | RMSNLAS | | 52 |
| VL CDR3 | MQHLEYPFT | | 57 |

### Example 2. Binding Activity of Anti-hCCR8 Chimeric Antibodies

To evaluate the binding activity of hybridoma clones 84D1-2H3, 86D4E12A5, 96G3-1F10, 99D1-1E11, 101D5G10G4, 115C5E3B8, 163H9D5, 187B5F10, 195H8D10 and 200C9B9, the chimeric mAbs from these clones were subjected to FACS test using HEK293 hCCR8 cell line, CHO-K1 cyno CCR8 cell line and CHO-K1 hCCR4 cell line.

Briefly, HEK293 hCCR8 cell line, CHO-K1 cyno CCR8 cell line or CHO-K1 hCCR4 cell line was first incubated with 5-fold serially diluted and 8 doses with the above 10 chimeric Abs and hlgG1 control starting from 100 nM at 4° C. for 30 mins. After washing by FACS buffer, PE Goat anti-Human IgG Fc Secondary Antibody (eBioscience™, Invitrogen) was added to each well and incubated at 4° C. for 30 mins. Samples were washed twice with FACS buffer. The mean florescence intensity (MFI) of PE was evaluated by MACSQuant Analyzer 16.

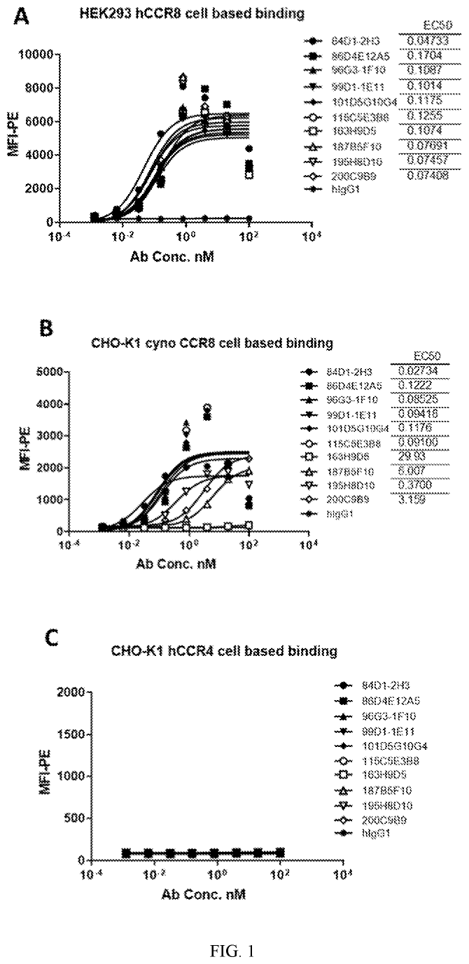

As shown in FIG. 1A, anti-hCCR8 antibodies all specifically bound to human CCR8 in a dose-dependent manner. As shown in FIG. 1B, all anti-hCCR8 antibodies except 163H9D5 bound to cyno CCR8 as well, although to a different extent. As shown in FIG. 1C, none of these antibodies bound to the closest family member CCR4, further suggesting these antibodies are specific to CCR8.

### Example 3. Blockade of hCCL1 Induced Signaling by Anti-CCR8 Chimeric Antibodies

It was reported that CCR8 ligand CCL1 was overexpressed and secreted into the cancer microenvironment by tumor associated macrophage, cancer associated fibroblast or other cell types. By activating its receptor CCR8, it causes the proliferation, apoptosis resistance and migration of immunosuppressive regulatory T cells, thus further inhibit the anti-tumor immunity. Therefore, anti-hCCR8 antibodies in a chimeric format were tested in cell-based assays for its ability to inhibit CCL1 mediated downstream signaling.

In the β-Arrestin assays, CCR8 is fused in frame with a transcription factor Gal4, linked by a protease site. Upon CCL1 binding, β-arrestin fused with a protease is recruited to the intracellular terminal and the tagged protease then cleaves the transcription factor which translocates to the nucleus and activates the expression of Gal4 dependent luciferase expression. Briefly, β-Arrestin hCCR8 CHO-K1 cells were plated in each well of 96-well assay plate at a density of 20000 per well, and cultured in 37° C., 5% CO₂. After 24 hours of incubation, each chimeric antibody was serially diluted and added to the designed rows of the assay plate. Then the final concentration 10 ng/ml of human CCL1 (R&D system) was then added and incubated for another 12-16 hours. Working detection solution (ONE-Glo™ Luciferase assay system from Promega) was added to all wells of the assay plate, and the assay plate was incubated for 3 mins at room temperature in the dark. The luminescence was read on Multimode Plate Reader (Envision® 2105).

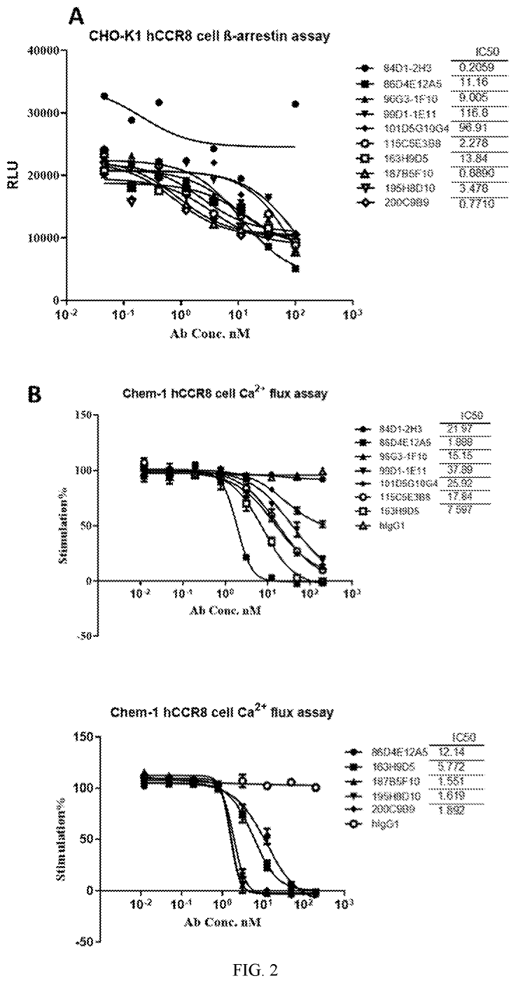

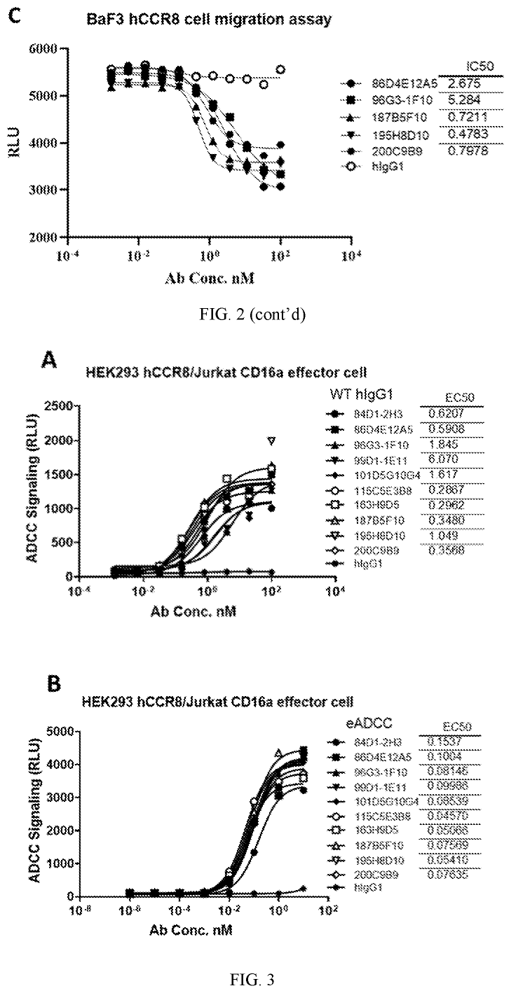

Data was plotted using GraphPad Prism software to obtain half-maximal inhibitory concentration (IC₅₀) values and summarized in FIG. 2A. As showed in FIG. 2A, all the antibodies except 84D1-2H3 showed varying levels of antagonism in the hCCL1 induced β-Arrestin recruitment to hCCR8 in CHO-K1 cell.

In addition, blockade of hCCL1 mediated pathway by anti-hCCR8 mAbs was tested by conducting calcium (Ca²⁺) flux assay on ChemiScreen™ CCR8 Chemokine receptor stable cell line (Chem1-hCCR8, Discover X) since hCCL1 engagement of CCR8 on Chem1 cells induced calcium flux. Chem-1 hCCR8 cells were seeded in 384-well assay plate in a density of 5000 per well and cultured in 37° C., 5% CO₂ for at least 18 hours. Dye working buffer (Screen Quest™ Fluo-8 No wash calcium assay kit, AAT Bioquest) and different anti-hCCR8 mAbs were added to the cells and incubated at 37° C., 5% CO₂ for 1 hour before the addition of 19 nM hCCL1 (R&D system). The Ca²⁺ flux fluorescent signal was measured by Max-Min using FLIPR™ TETRA (Molecular Device). The IC₅₀ was calculated using Graphpad Prism.

As showed in FIG. 2B, all antibodies except 84D1-2H3 showed varying levels of antagonism in the hCCL1 induced Ca²⁺ flux to hCCR8 in Chem1 cell, which is consistent with β-Arrestin assays.

Furthermore, the chemotaxis assay was performed to assess the inhibitory activity of 86D4E12A5, 96G3-1F10, 187B5F10, 195H8D10 and 200C9B9 on their ability to block CCL1-induced migration of CCR8 expressing cells. The BaF3 hCCR8 cell suspension and the diluted antibody samples were added to 96-well plate and incubated for 1 hour at 37° C., 5% CO₂. hCCL1 (80 μl) solution at a concentration of 40 ng/ml was transferred to corresponding wells in the bottom section of the chemotaxis chamber and assemble chamber using 5 μm pore size chemotaxis filter. Then the cell/antibody mixture (100 μl) were transferred to the top section of the chamber and incubated at 37° C. for 3 hours. Finally, the contents from the lower section of the chemotaxis chamber were transferred to a new 96 well plate and 10 μl resazurin was added to the plate. After incubation for 24 hours at 37° C., the plate was read in fluorescent plate reader at 544/590 nM.

FIG. 2C showed that 86D4E12A5, 96G3-1F10, 187B5F10, 195H8D10 and 200C9B9 all inhibited the migration of BaF3 hCCR8 induced by hCCL1.

### Example 4. Antibodies Induce ADCC Signaling in ADCC Reporter Bioassay

The example compared the ADCC signaling of CCR8 antibodies in vitro.

In this experiment, HEK293 hCCR8 cells were used as the target cells and Jurkat cells that stably expressing the high affinity FcγRIIIa [CD16a (176V)] and a luciferase reporter driven by an NFAT-response element were used as the effector cells and the tested antibodies were anti-hCCR8 antibodies with WT hlgG1 Fc or hlgG1 S239D/1332E Fc. Upon simultaneous engagement on both target cells and effector cells, effector cells will transduce intracellular signal, resulting in NFAT-mediated luciferase that can be quantified by a luminescence reader. In this experiment, target cells and effectors cells were mixed in an E:T ratio of 1 to 1, with addition of antibodies with ascending concentration and incubated in 96 well assay plates for 6 hours at 37° C., 5% CO₂. ADCC activity was determined by a bioluminescent readout.

The results (FIG. 3A) showed that the chimeric anti-hCCR8 antibodies with wildtype hIgG1, induced ADCC signaling in a dose-dependent manner.

As shown in FIG. 3B, the chimeric anti-hCCR8 antibodies with an enhanced Fc backbone (hlgG1 S239D/1332E) induced stronger ADCC signaling in the same experiment setting.

### Example 5. Antibodies Induce ADCC with HEK293 hCCR8 Cells Using NK92 CD16a as Effector Cells

Anti-hCCR8 antibodies with enhanced Fc (hlgG1 S239D/1332E) that exhibited stronger ADCC reporter activity in the reporter assay, were tested for their ability to mediate killing of target cells (HEK293 hCCR8 cell line) by a natural killer cytotoxic cell line NK-92 overexpressing CD16a (176V).

In this experiment, DELFIA® EuTDA Cytotoxicity kit (PerkinElmer) was utilized to measure ADCC. Target cells (HEK293 hCCR8 cells) was diluted to a density of 1,000,000 cells/ml with the culture medium and labelled with fluorescence enhancing ligand for 25 minutes at 37° C. After extensive wash for 3-5 times, labeled target cells with mixed with NK92 CD16a (176V) cells at a ratio of 1:10 and anti-hCCR8 antibodies with a series of concentrations, were added to microplate for coculture. After 2 hours' incubation, 20 μL supernatant were transferred to a new plate and add 200 μL of Europium solution and incubate for 15 minutes at room temperature using the DELFIA Plateshake. The signal correlates with the number of lysed cells.

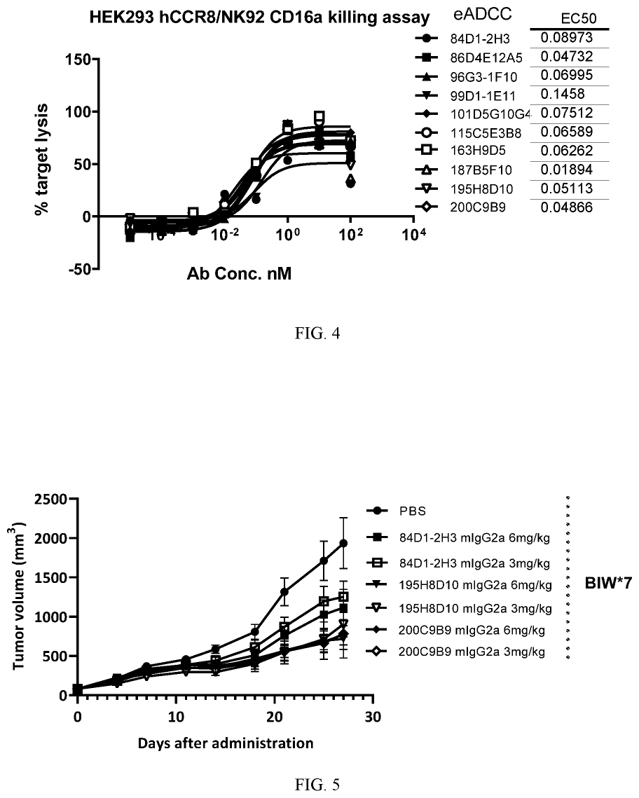

As shown in FIG. 4, the chimeric anti-hCCR8 antibodies with enhanced Fc function (hlgG1 S239D/1332E) could induce ADCC effect against CCR8 positive cells by natural killer cell line.

### Example 6. Efficacy in MC38 Tumor Model

This example used a syngeneic mouse model to test the in vivo anti-tumor efficacy of the functional molecules. The Fc of anti-CCR8 antibodies were engineered into mlgG2a to mimic the stronger ADCC effect of human IgG1.

MC38 cells resuspended in PBS were administered subcutaneously (s.c.) into the right flank of B-hCCR8 humanized C57BL/6 mice at a concentration of 5×10⁵ cells in a volume of 0.1 ml. When the average tumor volume reached approximately 82 mm³, the animals were randomly assigned to experimental groups according to the tumor volume, with 7 animals in each group. Anti-CCR8 antibodies were tested antibodies including 84D1-2H3-mIgG2a (6 mg/kg and 3 mg/kg), 195H8D10-mlgG2a (6 mg/kg and 3 mg/kg) and 200C9B9-mlgG2a (6 mg/kg and 3 mg/kg). The tested antibodies were administered twice every week by intraperitoneal injection. Mice body weight and tumor volume were measured twice a week.

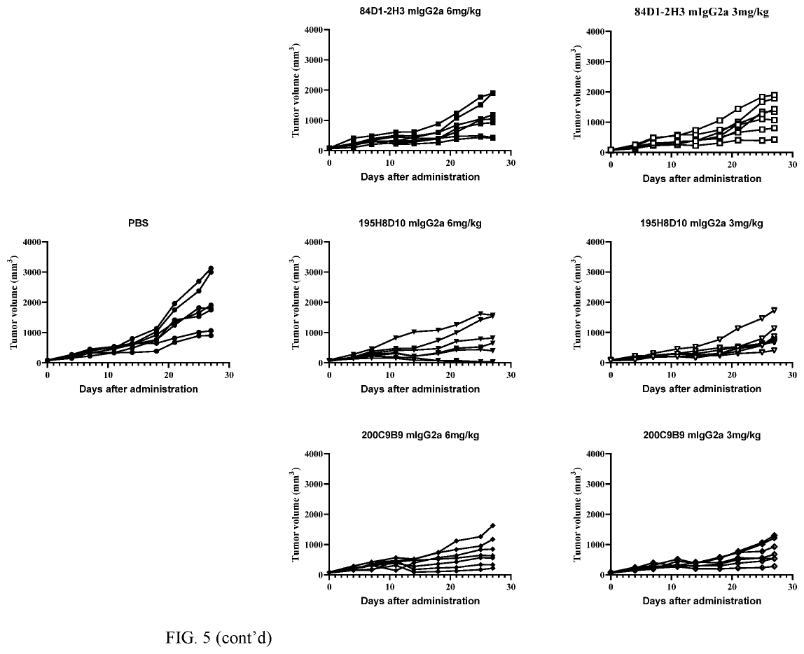

As shown in FIG. 5, all anti-human CCR8 antibodies with enhanced ADCC activity exhibited significant tumor growth inhibition both at a dose level of 3 mg/kg and 6 mg/kg. Among these antibodies, 195H8D10 and 200C9B9 showed stronger anti-tumor activity.

### Example 7. Humanization of the hCCR8 Antibodies

The variable region genes of antibodies 195H8D10 and 200C9B9 were employed to create humanized mAb. The amino acid sequences of the VH and VK of 195H8D10/200C9B9 were compared against the available database of human Ig gene sequences to find the overall best-matching human germline Ig sequences.

For the light chain of 195H8D10, IGKV2-28*01 is the best fit germline, and for the heavy chain of 195H8D10, IGHV3-72*01 was chosen as the humanization backbone. Humanized 195H8D10 CDR grafting antibody was then designed where the CDR-L1, L2, and L3 were grafted onto framework sequences of the IGKV2-28*01, and the CDR-H1, H2, and H3 were grafted onto framework sequences of the IGHV3-72*01. A 3D model was then generated to determine the amino acids in the original mouse framework region that are essential for antibody binding and conformation. Based on the analysis, back mutation was made on the grafting antibody, therefore, 4 additional humanized heavy chains and 4 additional light chains were generated

For the light chain of 200C9B9, IGKV2-28*01 is the best fit germline, and for the heavy chain of 200C9B9, IGHV3-72*01 was chosen as the humanization backbone. Humanized 200C9B9 CDR grafting antibody was then designed where the CDRL1, L2, and L3 were grafted onto framework sequences of the IGKV2-28*01, and the CDRH1, H2, and H3 were grafted onto framework sequences of the IGHV3-72*01. A 3D model was then generated to determine the amino acids in the original mouse FR region sequences that are essential for antibody binding and conformation. Based on the analysis, back mutation was made on the 200C9B9 CDR grafting antibody, 4 additional humanized heavy chains and 4 additional light chains were generated.

The sequences of the resulting humanized sequences are listed in Tables 2-3.

TABLE 2
Humanization of 195H8D10

| Name | Sequence | SEQ ID NO: |
|---|---|---|
| 195H8D10 VH | EVQLVGSGGGLVQPKGSLKLSCAASGFNFNTYVMNWVRQAPGKGLEWVAR | 17 |
| | IRTKSNNYATYYADSVKDRFTISRDDSESKLYLQMNNLKTEDTAMYYCVS | |
| | GTITRLGGGLDYWGQGTSVTVSS | |
| 195H8D10 | EVQLVESGGGLVQPGGSLRLSCAASGFNFNTYVMNWVRQAPGKGLEWVGR | 76 |
| VH.V1 (grafted) | IRTKSNNYATYYADSVKDRFTISRDDSKNSLYLQMNSLKTEDTAVYYCAR | |
| | GTITRLGGGLDYWGQGTLVTVSS | |
| 195H8D10 | EVQLVESGGGLVQPGGSLRLSCAASGFNFNTYVMNWVRQAPGKGLEWVAR | 77 |
| VH.V2 | IRTKSNNYATYYADSVKDRFTISRDDSKNSLYLQMNSLKTEDTAVYYCVS | |
| | GTITRLGGGLDYWGQGTLVTVSS | |
| 195H8D10 | EVQLVESGGGLVQPGGSLRLSCAASGFNFNTYVMNWVRQAPGKGLEWVAR | 78 |
| VH.V3 | IRTKSNNYATYYADSVKDRFTISRDDSKSSLYLQMNSLKTEDTAVYYCVS | |
| | GTITRLGGGLDYWGQGTLVTVSS | |
| 195H8D10 | EVQLVESGGGLVQPGGSLRLSCAASGFNFNTYVMNWVRQAPGKGLEWVAR | 79 |
| VH.V4 | IRTKSNNYATYYADSVKDRFTISRDDSESSLYLQMNSLKTEDTAMYYCVS | |
| | GTITRLGGGLDYWGQGTLVTVSS | |
| 195H8D10 VL | DIVMTQAAPSVPVTPGESVSISCRSSKRLLHSNGNTYLYWFLQRPGQSPQ | 18 |
| | LLIYRMSNLASGVPDRFSGSGSGTAFTLRISRVEAEDVGVYYCMQHLEYP | |
| | FTFGGGTKLEIK | |
| 195H8D10 | DIVMTQSPLSLPVTPGEPASISCRSSKRLLHSNGNTYLYWYLQKPGQSPQ | 80 |
| VL.V1 (grafted) | LLIYRMSNLASGVPDRFSGSGSGTDFTLKISRVEAEDVGVYYCMQHLEYP | |
| | FTFGGGTKLEIK | |
| 195H8D10 | DIVMTQSPLSLPVTPGEPASISCRSSKRLLHSNGNTYLYWFLQKPGQSPQ | 81 |
| VL.V2 | LLIYRMSNLASGVPDRFSGSGSGTDFTLKISRVEAEDVGVYYCMQHLEYP | |
| | FTFGGGTKLEIK | |
| 195H8D10 | DIVMTQSPLSLPVTPGEPASISCRSSKRLLHSNGNTYLYWYLQKPGQSPQ | 82 |
| VL.V3 | LLIYRMSNLASGVPDRFSGSGSGTAFTLKISRVEAEDVGVYYCMQHLEYP | |
| | FTFGGGTKLEIK | |
| 195H8D10 | DIVMTQSPLSLPVTPGEPASISCRSSKRLLHSNGNTYLYWFLQKPGQSPQ | 83 |
| VL.V4 | LLIYRMSNLASGVPDRFSGSGSGTAFTLKISRVEAEDVGVYYCMQHLEYP | |
| | FTFGGGTKLEIK | |

TABLE 2A
Humanized antibodies from 195H8D10

| | VL | | | |
|---|---|---|---|---|
| | 195H8D10 | 195H8D10 | 195H8D10 | 195H8D10 |
| VH | VL.V1 | VL.V2 | VL.V3 | VL.V4 |
| 195H8D10 VH.V1 | Hu195H8D10-1 | Hu195H8D10-2 | Hu195H8D10-3 | Hu195H8D10-4 |
| 195H8D10 VH.V2 | Hu195H8D10-5 | Hu195H8D10-6 | Hu195H8D10-7 | Hu195H8D10-8 |
| 195H8D10 VH.V3 | Hu195H8D10-9 | Hu195H8D10-10 | Hu195H8D10-11 | Hu195H8D10-12 |
| 195H8D10 VH.V4 | Hu195H8D10-13 | Hu195H8D10-14 | Hu195H8D10-15 | Hu195H8D10-16 |

TABLE 3
Humanization of 200C9B9

| Name | Sequence | SEQ ID NO: |
|---|---|---|
| 200C9B9 VH | EVQLVESGGGLVQPKGSLKLSCAASGFSFNTYVMNWVRQAPGKGLNWVAR | 19 |
| | IRTKSNNYATFYVDSVKDRFTISRDDSENMLYLQMNNLKIEDTAIYYCVS | |
| | GTITRLGAGLDYWGQGTSVTVSS | |
| 200C9B9 | EVQLVESGGGLVQPGGSLRLSCAASGFSFNTYVMNWVRQAPGKGLEWVGR | 84 |
| VH.V1 (grafted) | IRTKSNNYATFYVDSVKDRFTISRDDSKNSLYLQMNSLKTEDTAVYYCAR | |
| | GTITRLGAGLDYWGQGTLVTVSS | |
| 200C9B9 | EVQLVESGGGLVQPGGSLRLSCAASGFSFNTYVMNWVRQAPGKGLEWVAR | 85 |
| VH.V2 | IRTKSNNYATFYVDSVKDRFTISRDDSKNSLYLQMNSLKTEDTAVYYCVS | |
| | GTITRLGAGLDYWGQGTLVTVSS | |
| 200C9B9 | EVQLVESGGGLVQPGGSLRLSCAASGFSFNTYVMNWVRQAPGKGLNWVAR | 86 |
| VH.V3 | IRTKSNNYATFYVDSVKDRFTISRDDSKNSLYLQMNSLKTEDTAVYYCVS | |
| | GTITRLGAGLDYWGQGTLVTVSS | |
| 200C9B9 | EVQLVESGGGLVQPGGSLRLSCAASGFSFNTYVMNWVRQAPGKGLNWVAR | 87 |
| VH.V4 | IRTKSNNYATFYVDSVKDRFTISRDDSENSLYLQMNSLKTEDTAVYYCVS | |
| | GTITRLGAGLDYWGQGTLVTVSS | |
| 200C9B9 VL | DIVMTQAAPSVPVTPGESVSISCRSSKSLLHSNGNTYLYWFLQRPGQSPQ | 20 |
| | LLIYRMSNLASGVPDRFSGSGSGTAFTLRISRVEAEDVGVYYCMQHLEYP | |
| | FTFGGGTKLEIK | |
| 200C9B9 | DIVMTQSPLSLPVTPGEPASISCRSSKSLLHSNGNTYLYWYLQKPGQSPQ | 88 |
| VL.V1 (grafted) | LLIYRMSNLASGVPDRFSGSGSGTDFTLKISRVEAEDVGVYYCMQHLEYP | |
| | FTFGGGTKLEIK | |
| 200C9B9 | DIVMTQSPLSLPVTPGEPASISCRSSKSLLHSNGNTYLYWFLQKPGQSPQ | 89 |
| VL.V2 | LLIYRMSNLASGVPDRFSGSGSGTDFTLKISRVEAEDVGVYYCMQHLEYP | |
| | FTFGGGTKLEIK | |
| 200C9B9 | DIVMTQSPLSLPVTPGEPASISCRSSKSLLHSNGNTYLYWYLQKPGQSPQ | 90 |
| VL.V3 | LLIYRMSNLASGVPDRFSGSGSGTAFTLKISRVEAEDVGVYYCMQHLEYP | |
| | FTFGGGTKLEIK | |
| 200C9B9 | DIVMTQSPLSLPVTPGEPASISCRSSKSLLHSNGNTYLYWFLQKPGQSPQ | 91 |
| VL.V4 | LLIYRMSNLASGVPDRFSGSGSGTAFTLKISRVEAEDVGVYYCMQHLEYP | |
| | FTFGGGTKLEIK | |

TABLE 3A
Humanized antibodies from 200C9B9

| | VL | | | |
|---|---|---|---|---|
| | 200C9B9 | 200C9B9 | 200C9B9 | 200C9B9 |
| VH | VL.V1 | VL.V2 | VL.V3 | VL.V4 |
| 200C9B9 VH.V1 | Hu200C9B9-1 | Hu200C9B9-2 | Hu200C9B9-3 | Hu200C9B9-4 |
| 200C9B9 VH.V2 | Hu200C9B9-5 | Hu200C9B9-6 | Hu200C9B9-7 | Hu200C9B9-8 |
| 200C9B9 VH.V3 | Hu200C9B9-9 | Hu200C9B9-10 | Hu200C9B9-11 | Hu200C9B9-12 |
| 200C9B9 VH.V4 | Hu200C9B9-13 | Hu200C9B9-14 | Hu200C9B9-15 | Hu200C9B9-16 |

### Example 8. Binding Activity of the Humanized Antibodies

Flow cytometry was used to confirm the binding activity of the humanized antibodies using CHO-K1 hCCR8 cell line and CHO-K1 cyno CCR8 cell line.

Briefly, CHO-K1 hCCR8 cells and CHO-K1 cyno CCR8 cells were firstly incubated with humanized 195H8D10 and 200C9B9 antibodies of different concentrations at 4° C. for 30 mins. PE Goat anti-Human IgG Fc Secondary Antibody (eBioscience™, Invitrogen) was added to each well and incubated at 4° C. for 30 mins. Samples were washed twice with FACS buffer. The mean florescence intensity (MFI) of PE was evaluated by MACSQuant Analyzer 16.

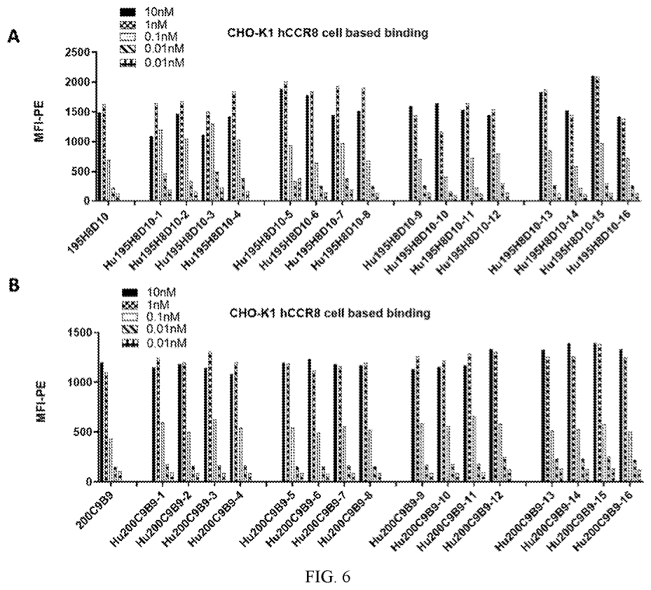

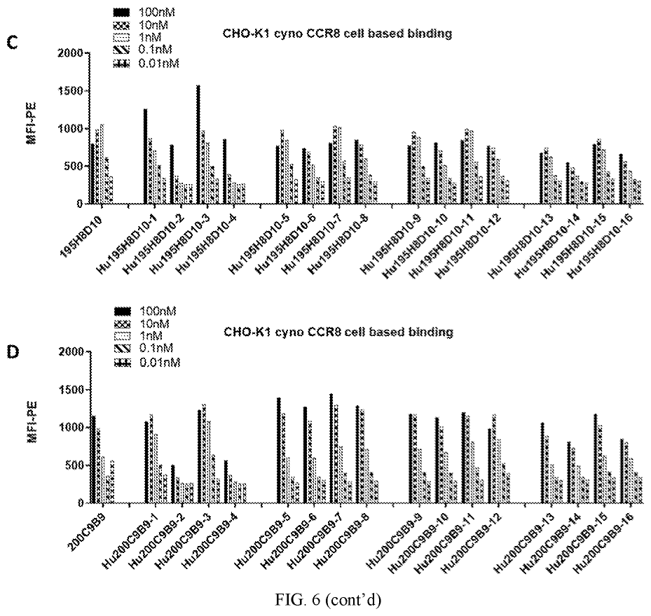

As shown in FIGS. 6A and 6B, the humanized 195H8D10/200C9B9 antibodies were comparable to the corresponding chimeric antibodies on human CCR8 binding.

Furthermore, FIGS. 6C and 6D showed that the humanized 195H8D10/200C9B9 antibodies except Hu195H8D10-2, Hu195H8D10-4, Hu200C9B9-2, and Hu200C9B9-4, were comparable to the corresponding chimeric antibodies on cyno CCR8 binding.

### Example 9. Humanized Antibodies Induce ADCC Signaling in ADCC Reporter Bioassay

The example compared the ADCC signaling of the humanized CCR8 antibodies in vitro.

In this experiment, as stated in the previous Example, CHO-K1 hCCR8 cells were used as the target cells, Jurkat CD16a (176V) NFAT Luciferase receptor cells were used as the effector cells and the tested antibodies were humanized 195H8D10 and 200C9B9 antibodies. The experiment was carried out under the co-incubation of target cells, effector cells and testing antibodies in 96 well assay plates for 6 hours at 37° C., 5% CO₂ according to manufacturer's instructions. ADCC signaling was determined by a bioluminescent readout.

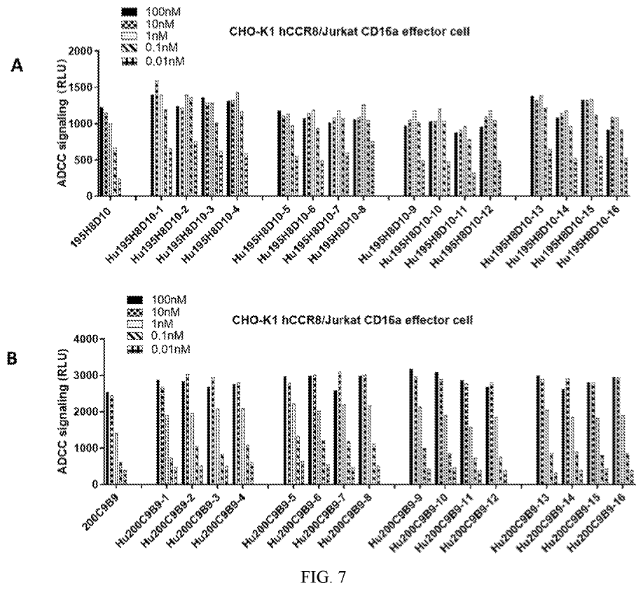

As shown in FIGS. 7A and 7B, humanized 195H8D10/200C9B9 antibodies induced comparable ADCC signaling to the corresponding chimeric antibodies in the ADCC reporter assay.

### Example 10. Inhibition of hCCL1 Induced β-Arrestin Recruitment by Humanized Antibodies

The humanized 195H8D10/200C9B9 antibodies were again tested to confirm the antagonism of CCL1 mediated downstream signaling on CHO-K1 hCCR8 cells.

The experiment was done as described in the previous Example. Briefly, β-Arrestin hCCR8 CHO-K1 cells were plated and mixed with diluted testing antibodies. Human CCL1 (R&D system) was then added to the culture and incubated for 12-16 hours. Detection buffer was added to all wells of the assay plate, and luminescence was read on a microplate reader.

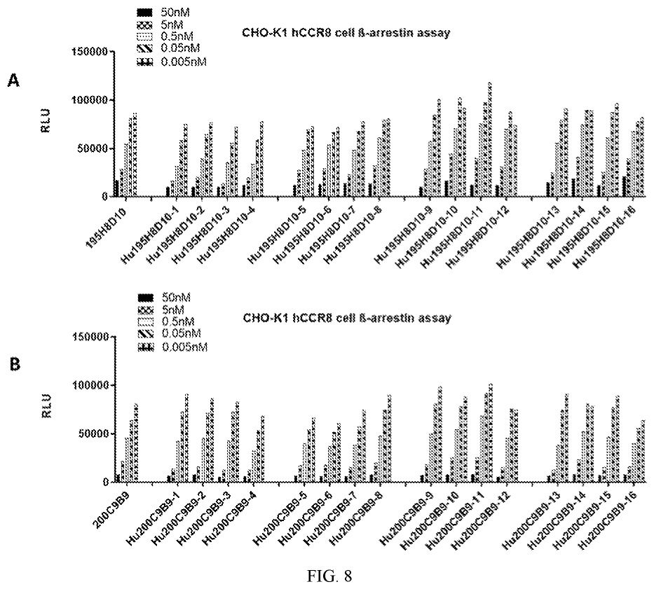

As showed in FIGS. 8A and 8B, all the humanized 195H8D10/200C9B9 antibodies showed comparable levels of antagonism to the corresponding chimeric antibody in the hCCL1 induced β-Arrestin assay.

### Example 11. PTM Removal Confirm of 195H8D10 and 200C9B9

It has been observed that the 195H8D10 and 200C9B9 VL CDR1 include NG residues (Kabat numbering) which are at risk of post-translational modifications (PTM) and pose challenges for future manufacturing. Therefore, this example mutated NG to QG or NA on the VL to prevent PTM. The sequences of the potential PTM removal site are listed in Table 4.

TABLE 4
PTM removal of 195H8D10 and 200C9B9

| | VL | | | | | |
|---|---|---|---|---|---|---|
| | 195H8D10 | 195H8D10 | 195H8D10 | 200C9B9 | 200C9B9 | 200C9B9 |
| VH | VL | VL NG-QG | VL NG-NA | VL | VL NG-QG | VL NG-NA |
| 195H8D10 VH | 195H8D10- | 195H8D10- | 195H8D10- | | | |
| | C | C1 | C2 | | | |
| 200C9B9 VH | | | | 200C9B9- | 200C9B9- | 200C9B9- |
| | | | | C | C1 | C2 |

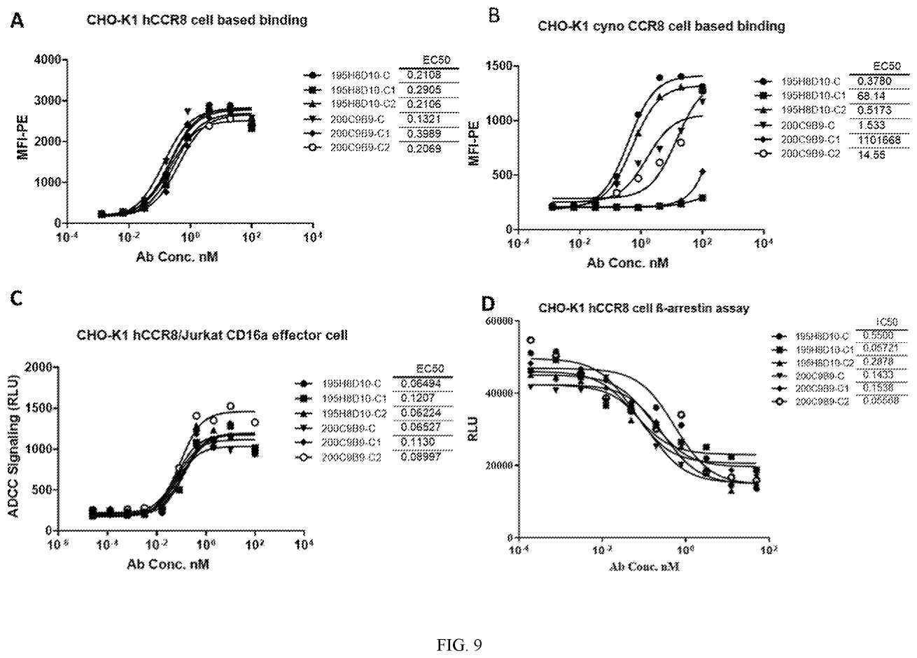

As showed in FIG. 9A, 195H8D10-C1/C2 and 200C9B9-C1/C2 were comparable to the corresponding parental antibodies on human CCR8 binding.

As showed in FIG. 9B, 195H8D10-C2 and 200C9B9-C2 maintain cyno binding capacity compared with the corresponding antibodies.

As showed in FIG. 9C, 195H8D10-C1/C2 and 200C9B9-C1/C2 induced comparable ADCC signaling to the corresponding parental antibodies in the ADCC reporter assay.

As showed in FIG. 9D, 195H8D10-C1/C2 and 200C9B9-C1/C2 showed comparable levels of antagonism to the corresponding parental antibodies in the hCCL1 induced β-Arrestin recruitment to hCCR8 in CHO-K1 cell.

This example therefore confirmed the sequences of humanized 195H8D10 and 200C9B9 with removal of potential PTM site (VL CDR1 NG-NA) and sequences are listed in Table 5.

TABLE 5
Humanization of 195H8D10 with removal of potential PTM site

| Name | Sequence | SEQ ID NO: |
|---|---|---|
| 195H8D10 VH | EVQLVGSGGGLVQPKGSLKLSCAASGFNFNTYVMNWVRQAPGKGLEWVAR | 17 |
| | IRTKSNNYATYYADSVKDRFTISRDDSESKLYLQMNNLKTEDTAMYYCVS | |
| | GTITRLGGGLDYWGQGTSVTVSS | |
| 195H8D10 | EVQLVESGGGLVQPGGSLRLSCAASGFNFNTYVMNWVRQAPGKGLEWVGR | 76 |
| VH.V1 (grafted) | IRTKSNNYATYYADSVKDRFTISRDDSKNSLYLQMNSLKTEDTAVYYCAR | |
| | GTITRLGGGLDYWGQGTLVTVSS | |
| 195H8D10 | EVQLVESGGGLVQPGGSLRLSCAASGFNFNTYVMNWVRQAPGKGLEWVAR | 77 |
| VH.V2 | IRTKSNNYATYYADSVKDRFTISRDDSKNSLYLQMNSLKTEDTAVYYCVS | |
| | GTITRLGGGLDYWGQGTLVTVSS | |
| 195H8D10 | EVQLVESGGGLVQPGGSLRLSCAASGFNFNTYVMNWVRQAPGKGLEWVAR | 78 |
| VH.V3 | IRTKSNNYATYYADSVKDRFTISRDDSKSSLYLQMNSLKTEDTAVYYCVS | |
| | GTITRLGGGLDYWGQGTLVTVSS | |
| 195H8D10 | EVQLVESGGGLVQPGGSLRLSCAASGFNFNTYVMNWVRQAPGKGLEWVAR | 79 |
| VH.V4 | IRTKSNNYATYYADSVKDRFTISRDDSESSLYLQMNSLKTEDTAMYYCVS | |
| | GTITRLGGGLDYWGQGTLVTVSS | |
| 195H8D10 VL | DIVMTQAAPSVPVTPGESVSISCRSSKRLLHSNGNTYLYWFLQRPGQSPQ | 18 |
| | LLIYRMSNLASGVPDRFSGSGSGTAFTLRISRVEAEDVGVYYCMQHLEYP | |
| | FTFGGGTKLEIK | |
| 195H8D10 | DIVMTQSPLSLPVTPGEPASISCRSSKRLLHSNANTYLYWYLQKPGQSPQ | 92 |
| VL.V1 (grafted) | LLIYRMSNLASGVPDRFSGSGSGTDFTLKISRVEAEDVGVYYCMQHLEYP | |
| NG-NA | FTFGGGTKLEIK | |
| 195H8D10 | DIVMTQSPLSLPVTPGEPASISCRSSKRLLHSNANTYLYWFLQKPGQSPQ | 93 |
| VL.V2 NG-NA | LLIYRMSNLASGVPDRFSGSGSGTDFTLKISRVEAEDVGVYYCMQHLEYP | |
| | FTFGGGTKLEIK | |
| 195H8D10 | DIVMTQSPLSLPVTPGEPASISCRSSKRLLHSNANTYLYWYLQKPGQSPQ | 94 |
| VL.V3 NG-NA | LLIYRMSNLASGVPDRFSGSGSGTAFTLKISRVEAEDVGVYYCMQHLEYP | |
| | FTFGGGTKLEIK | |
| 195H8D10 | DIVMTQSPLSLPVTPGEPASISCRSSKRLLHSNANTYLYWFLQKPGQSPQ | 95 |
| VL.V4 NG-NA | LLIYRMSNLASGVPDRFSGSGSGTAFTLKISRVEAEDVGVYYCMQHLEYP | |
| | FTFGGGTKLEIK | |

TABLE 5A
195H8D10 Humanized antibodies with removal of potential PTM site

| | VL | | | |
|---|---|---|---|---|
| | 195H8D10 | 195H8D10 | 195H8D10 | 195H8D10 |
| VH | VL.V1 NG-NA | VL.V2 NG-NA | VL.V3 NG-NA | VL.V4 NG-NA |
| 195H8D10 VH.V1 | Hu195H8D10- | Hu195H8D10- | Hu195H8D10- | Hu195H8D10- |
| | 1 NG-NA | 2 NG-NA | 3 NG-NA | 4 NG-NA |
| 195H8D10 VH.V2 | Hu195H8D10- | Hu195H8D10- | Hu195H8D10- | Hu195H8D10- |
| | 5 NG-NA | 6 NG-NA | 7 NG-NA | 8 NG-NA |
| 195H8D10 VH.V3 | Hu195H8D10- | Hu195H8D10- | Hu195H8D10- | Hu195H8D10- |
| | 9 NG-NA | 10 NG-NA | 11 NG-NA | 12 NG-NA |
| 195H8D10 VH.V4 | Hu195H8D10- | Hu195H8D10- | Hu195H8D10- | Hu195H8D10- |
| | 13 NG-NA | 14 NG-NA | 15 NG-NA | 16 NG-NA |

TABLE 6
Humanization of 200C9B9 with removal of potential PTM site

| Name | Sequence | SEQ ID NO: |
|---|---|---|
| 200C9B9 VH | EVQLVESGGGLVQPKGSLKLSCAASGFSFNTYVMNWVRQAPGKGLNWVAR | 19 |
| | IRTKSNNYATFYVDSVKDRFTISRDDSENMLYLQMNNLKIEDTAIYYCVS | |
| | GTITRLGAGLDYWGQGTSVTVSS | |
| 200C9B9 | EVQLVESGGGLVQPGGSLRLSCAASGFSFNTYVMNWVRQAPGKGLEWVGR | 84 |
| VH.V1 (grafted) | IRTKSNNYATFYVDSVKDRFTISRDDSKNSLYLQMNSLKTEDTAVYYCAR | |
| | GTITRLGAGLDYWGQGTLVTVSS | |
| 200C9B9 | EVQLVESGGGLVQPGGSLRLSCAASGFSFNTYVMNWVRQAPGKGLEWVAR | 85 |
| VH.V2 | IRTKSNNYATFYVDSVKDRFTISRDDSKNSLYLQMNSLKTEDTAVYYCVS | |
| | GTITRLGAGLDYWGQGTLVTVSS | |
| 200C9B9 | EVQLVESGGGLVQPGGSLRLSCAASGFSFNTYVMNWVRQAPGKGLNWVAR | 86 |
| VH.V3 | IRTKSNNYATFYVDSVKDRFTISRDDSKNSLYLQMNSLKTEDTAVYYCVS | |
| | GTITRLGAGLDYWGQGTLVTVSS | |
| 200C9B9 | EVQLVESGGGLVQPGGSLRLSCAASGFSFNTYVMNWVRQAPGKGLNWVAR | 87 |
| VH.V4 | IRTKSNNYATFYVDSVKDRFTISRDDSENSLYLQMNSLKTEDTAVYYCVS | |
| | GTITRLGAGLDYWGQGTLVTVSS | |
| 200C9B9 VL | DIVMTQAAPSVPVTPGESVSISCRSSKSLLHSNGNTYLYWFLQRPGQSPQ | 20 |
| | LLIYRMSNLASGVPDRFSGSGSGTAFTLRISRVEAEDVGVYYCMQHLEYP | |
| | FTFGGGTKLEIK | |
| 200C9B9 | DIVMTQSPLSLPVTPGEPASISCRSSKSLLHSNANTYLYWYLQKPGQSPQ | 96 |
| VL.V1 (grafted) | LLIYRMSNLASGVPDRFSGSGSGTDFTLKISRVEAEDVGVYYCMQHLEYP | |
| NG-NA | FTFGGGTKLEIK | |
| 200C9B9 | DIVMTQSPLSLPVTPGEPASISCRSSKSLLHSNANTYLYWFLQKPGQSPQ | 97 |
| VL.V2 NG-NA | LLIYRMSNLASGVPDRFSGSGSGTDFTLKISRVEAEDVGVYYCMQHLEYP | |
| | FTFGGGTKLEIK | |
| 200C9B9 | DIVMTQSPLSLPVTPGEPASISCRSSKSLLHSNANTYLYWYLQKPGQSPQ | 98 |
| VL.V3 NG-NA | LLIYRMSNLASGVPDRFSGSGSGTAFTLKISRVEAEDVGVYYCMQHLEYP | |
| | FTFGGGTKLEIK | |
| 200C9B9 | DIVMTQSPLSLPVTPGEPASISCRSSKSLLHSNANTYLYWFLQKPGQSPQ | 99 |
| VL.V4 NG-NA | LLIYRMSNLASGVPDRFSGSGSGTAFTLKISRVEAEDVGVYYCMQHLEYP | |
| | FTFGGGTKLEIK | |

TABLE 6A
Humanized antibodies from 200C9B9 with PTM removal

| | VL | | | |
|---|---|---|---|---|
| | 200C9B9 VL.V1 | 200C9B9 VL.V2 | 200C9B9 VL.V3 | 200C9B9 VL.V4 |
| VH | NG-NA | NG-NA | NG-NA | NG-NA |
| 200C9B9 | Hu200C9B9-1 | Hu200C9B9-2 | Hu200C9B9-3 | Hu200C9B9-4 |
| VH.V1 | NG-NA | NG-NA | NG-NA | NG-NA |
| 200C9B9 | Hu200C9B9-5 | Hu200C9B9-6 | Hu200C9B9-7 | Hu200C9B9-8 |
| VH.V2 | NG-NA | NG-NA | NG-NA | NG-NA |
| 200C9B9 | Hu200C9B9-9 | Hu200C9B9-10 | Hu200C9B9-11 | Hu200C9B9-12 |
| VH.V3 | NG-NA | NG-NA | NG-NA | NG-NA |
| 200C9B9 | Hu200C9B9-13 | Hu200C9B9-14 | Hu200C9B9-15 | Hu200C9B9-16 |
| VH.V4 | NG-NA | NG-NA | NG-NA | NG-NA |

### Example 12. PTM Removal Confirm of Humanized 195H8D10 and 200C9B9

To further confirm the performance of the humanized 195H8D10/200C9B9 antibodies after PTM removal, we tested the binding, ADCC activity, functional blocking activity, and tumor growth inhibition efficacy of the Hu195H8D10-1/7 NG-NA and the Hu200C9B9-3/7 NG-NA antibodies.

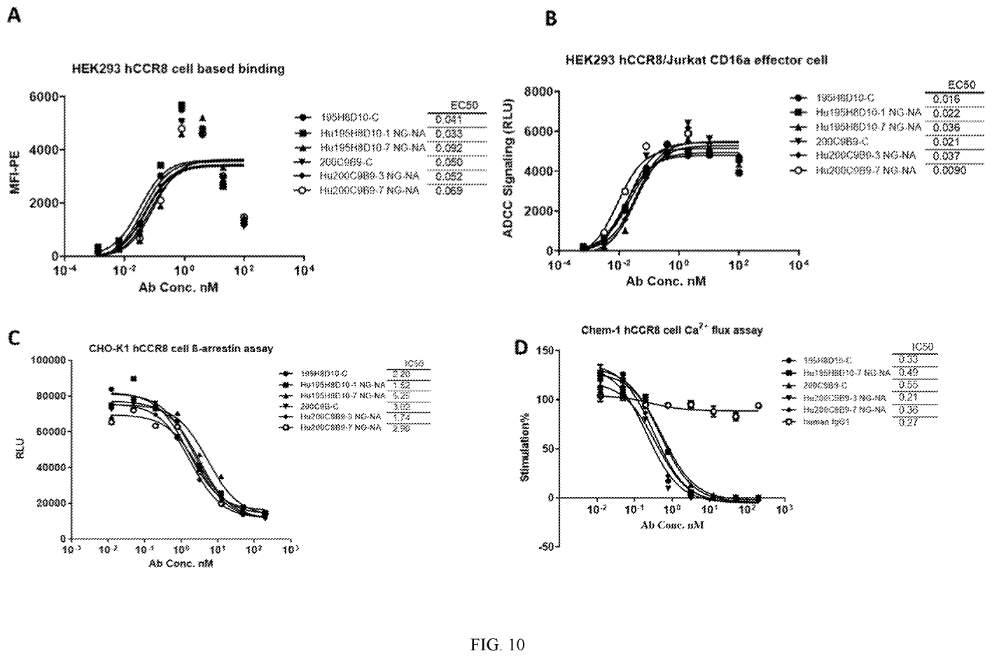

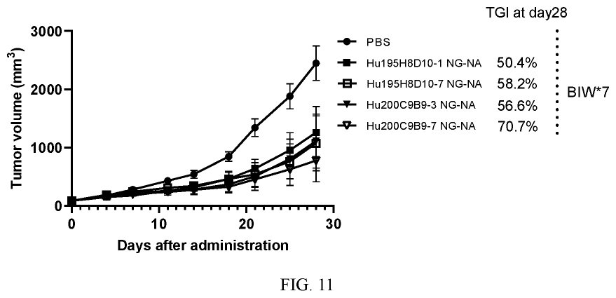

As showed in FIG. 10A, the Hu195H8D10-1/7 NG-NA and the Hu200C9B9-3/7 NG-NA antibodies were comparable to the corresponding parental antibodies on human CCR8 binding.

As showed in FIG. 10B, the Hu195H8D10-1/7 NG-NA and the Hu200C9B9-3/7 NG-NA antibodies induced comparable ADCC signaling to the corresponding parental antibodies in the ADCC reporter assay.

As showed in FIG. 10C, the Hu195H8D10-1/7 NG-NA and the Hu200C9B9-3/7 NG-NA antibodies showed comparable levels of antagonism to the corresponding parental antibodies in the hCCL1 induced β-Arrestin recruitment to hCCR8 in CHO-K1 cell.

As showed in FIG. 10D, the Hu195H8D10-7 NG-NA and the Hu200C9B9-3/7 NG-NA antibodies showed comparable levels of antagonism in the hCCL1 induced Ca²⁺ flux to hCCR8 in Chem1 cell, which is consistent with β-Arrestin assays.

Furthermore, we used a syngeneic mouse model to test the in vivo anti-tumor efficacy of the Hu195H8D10-1/7 NG-NA and the Hu200C9B9-3/7 NG-NA antibodies. The same mouse Fc was used for ADCC mediated Treg killing in the B-hCCR8 mice. Briefly, MC38 cells resuspended in PBS were administered subcutaneously (s.c.) into the right flank of B-hCCR8 humanized C57BL/6 mice at a concentration of 5×10⁵ cells in a volume of 0.1 ml. When the average tumor volume reached approximately 85 mm³, the animals were randomly assigned to experimental groups according to the tumor volume, with 7 animals in each group. Anti-CCR8 antibodies (6 mg/kg) were tested antibodies and administered twice every week by intraperitoneal injection. Mice body weight and tumor volume were measured twice a week.

As shown in FIG. 11, the Hu195H8D10-1/7 NG-NA and the Hu200C9B9-3/7 NG-NA antibodies with enhanced ADCC activity exhibited significant tumor growth inhibition (TGI). Among these antibodies, Hu200C9B9-7 NG-NA mono-treatment showed stronger inhibition of tumor growth with a inhibition rate around 70% at the end of the study.

### Example 13. Comparison of Hu200C9B9-7 NG-NA with Benchmark Antibodies

Finally, we compared the Hu200C9B9-7 NG-NA antibody with benchmark antibodies from Gilead Sciences (1-K17.044) and Bristol Myers Squibb (4A19) in vitro and in vivo. Anti-CCR8 antibodies developed by Gilead and BMS are currently in clinical trial of Phase I and Phase I/II, respectively.

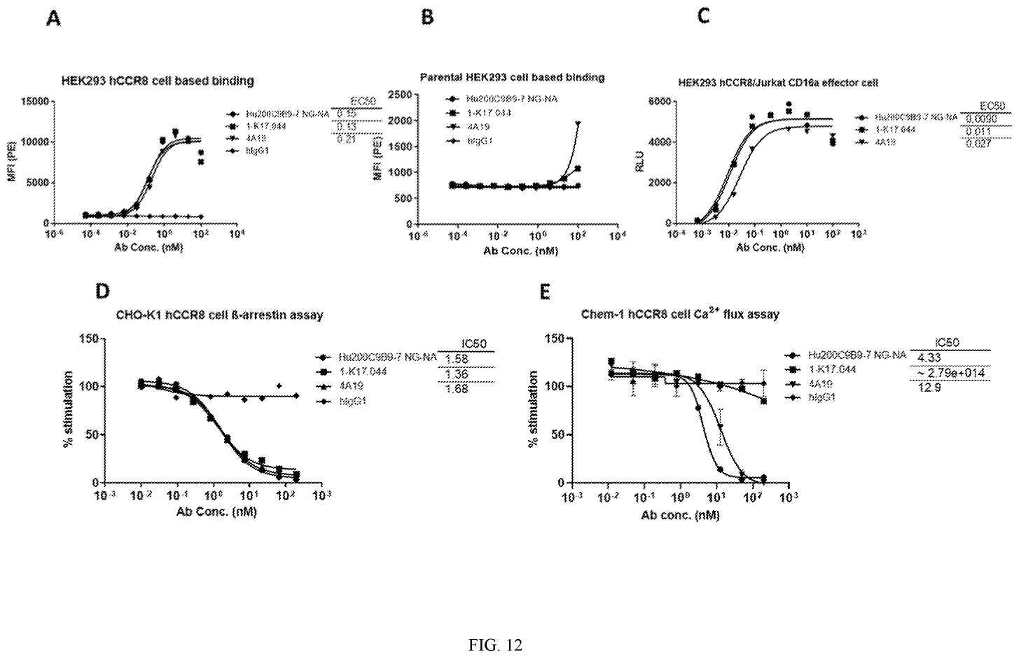

All CCR8 antibodies were fused with the same Fc (human IgG1 kappa). The binding activity, ADCC activity, and functional blocking activity were compared in vitro. As shown in FIG. 12A, Hu200C9B9-7 NG-NA was a strong binder of hCCR8 with binding capacity comparable to that of its benchmarks determined by FACS. To determine the specificity, we performed the binding assay based on the parental HEK293 without target. The results (FIG. 12B) showed Hu200C9B9-7 NG-NA did not bind the parental HEK293 at all while benchmark antibodies developed from Gilead and BMS had some degree of non-specific binding to parental HEK293 cell.

As stated in the previous example, we also compared the ADCC signaling of the Hu200C9B9-7 NG-NA and benchmarks. FIG. 12C showed that all the anti-hCCR8 antibodies with wildtype hlgG1 induced ADCC signaling in a dose-dependent manner in the HEK293 hCCR8 and Jurkat CD16a (176V) NFAT Luciferase cell coculture assay.

In addition, blockade of hCCL1 mediated pathway by Hu200C9B9-7 NG-NA and benchmark antibodies were tested by conducting β-arrestin recruitment assay and calcium (Ca²⁺) flux assay. Hu200C9B9-7 NG-NA showed comparable level of antagonism with benchmarks in the hCCL1 induced β-Arrestin recruitment to hCCR8 in CHO-K1 cell (FIG. 12D), while Hu200C9B9-7 NG-NA could better inhibit hCCL1 mediated Ca²⁺ flux in Chem-1 hCCR8 cell than both benchmark antibodies (FIG. 12E).

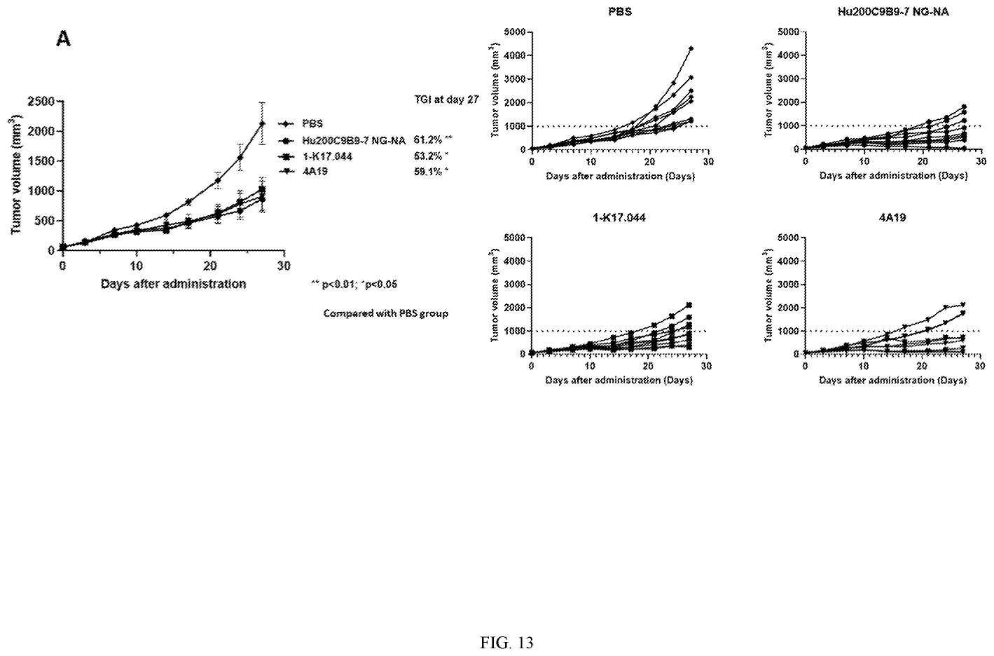

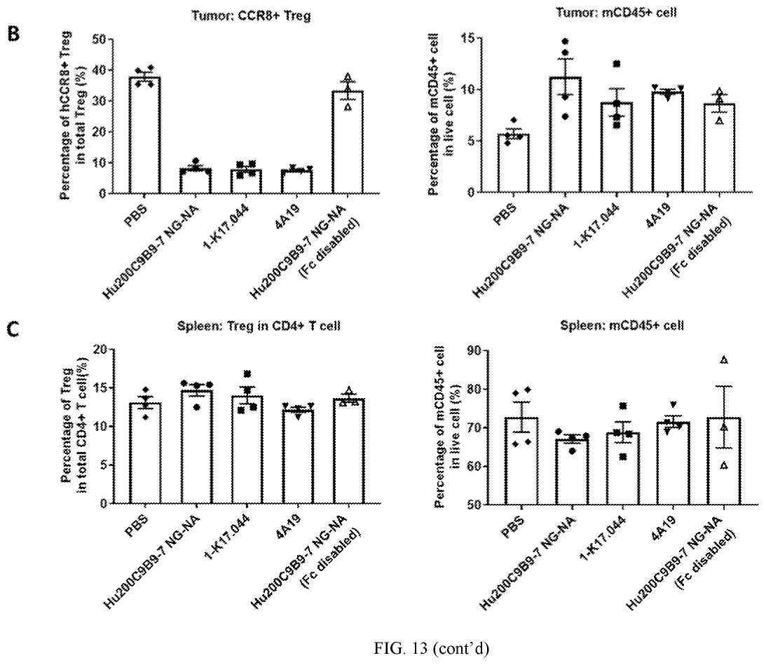

The preclinical efficacy of Hu200C9B9-7 NG-NA and benchmarks were evaluated in the syngeneic mouse model where the murine CCR8 gene was replaced by its human counterpart and MC38 was inoculated as described in the previous example. Using the same mouse Fc, Hu200C9B9-7 NG-NA, 1-K17.044 and 4A19 mono-treatment all showed significant inhibition of tumor growth with a inhibition rate 61.2%, 53.2% and 59.1%, respectively (FIG. 13A). Ex vivo analysis suggested all these antibodies efficiently depleted CCR8+ Tregs and increased mCD45+ lymphocytes in the tumor site (FIG. 13B) while sparing peripheral Treg subset and mCD45+ lymphocytes (FIG. 13C).

### Example 14. Fc Selection of Hu200C9B9-7 NG-NA

Fc engineering is a promising approach to enhance the ADCC effect for better antitumor efficacy of monoclonal antibodies (mAbs).

To find the best ADCC enhancing approach for therapeutic antibodies, we produced antibodies with Fc-DLE (introducing the triple mutation S239D/A330L/1332E to both Fc chains), Fc-DE (introducing the dual mutation S239D/1332E to both Fc chains), Fc-Afucosylated in which the N-glycan residues in the IgG Fc region missing core fucose sugar units, Fc-Asymmetric (i.e., introducing different substitutions into each Fc region of heavy chain: one heavy chain: L234Y/L235Q/G236W/S239M/H268D/D270E/S298A and opposing heavy chain: D270E/K326D/A330M/K334E) and Fc-WT hlgG1. We evaluated the binding affinity of these Fc variants or Glyco-mutant Fc against FcγRIIIa allotypes and inhibitory FcγRIIb, and the ratio of activating FcγR binding to inhibitory FcγR binding (A/I) was calculated.

The binding of the antibodies with different Fc to recombinant hCD16a-176V, hCD16a-176F and hCD32b protein were tested with Biacore using a capture method. The protein was captured using CM5-Anti-His chip. A serial dilution of antibodies were injected over captured protein. All the experiments were carried out on a Biacore T200. Data analysis was carried out using Biacore T200 evaluation software. The results are shown in Table 7. Fc variant with DLE mutation was found to have the high affinity to both CD16a allotypes and have the lowest affinity to CD32b. Thus, Fc variant with DLE mutations reached the highest A/I ratio.

TABLE 7
Affinity measured by Biacore and A/I ratio

| | | | Fc- | Fc- | |
|---|---|---|---|---|---|
| | Fc-DLE | Fc-DE | Afucosylated | Asymmetric | Fc-WT |
| A/I Ratio(hCD16a- | 407.9 | 0.8 | 159.6 | 12.4 | 1.3 |
| 176V/hCD32b) | | | | | |
| A/I Ratio(hCD16a- | 386.1 | 1.2 | 29.5 | 6.4 | 0.6 |
| 176F/hCD32b) | | | | | |
| Affinity to hCD16a- | 5.82 × 10⁻¹⁰ | 3.44 × 10⁻¹⁰ | 4.29 × 10⁻¹⁰ | 6.15 × 10⁻¹⁰ | 1.13 × 10⁻⁸ |
| 176V (high affinity - | | | | | |
| 40% population) | | | | | |
| (KD, M) | | | | | |
| Affinity to hCD16a- | 6.15 × 10⁻¹⁰ | 2.09 × 10⁻¹⁰ | 2.32 × 10⁻⁹ | 1.19 × 10⁻⁹ | 2.53 × 10⁻⁸ |
| 176F (low affinity - | | | | | |
| 60% population) | | | | | |
| (KD, M) | | | | | |
| Affinity to hCD32b | 2.38 × 10⁻⁷ | 2.44 × 10⁻¹⁰ | 6.84 × 10⁻⁸ | 7.62 × 10⁻⁹ | 1.46 × 10⁻⁸ |
| affinity (KD, M) | | | | | |

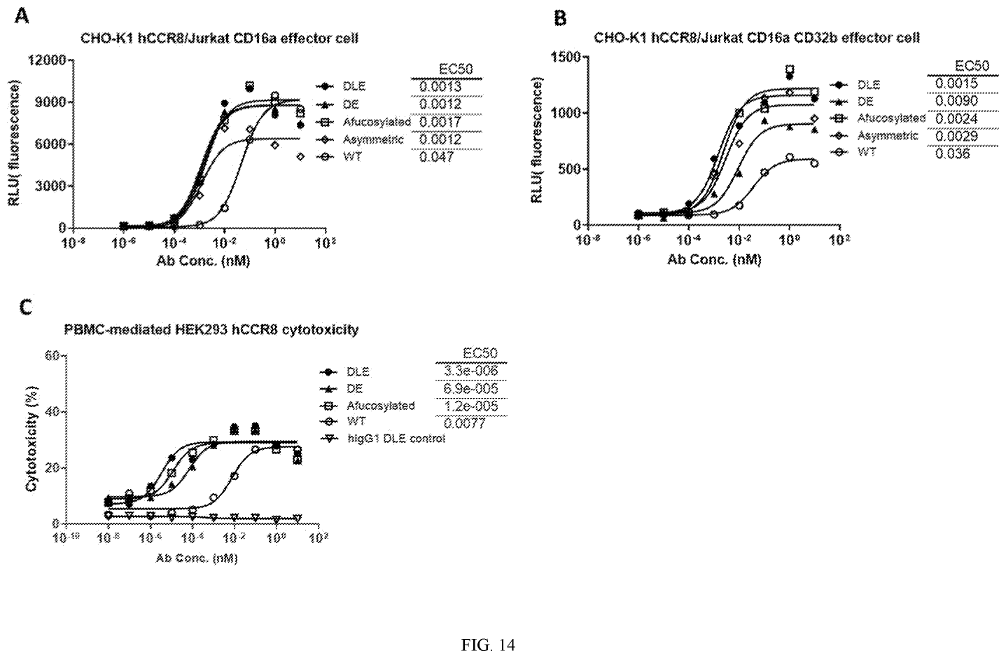

To further assess the ability of the CCR8 antibodies in ADCC signaling, we compared the anti-CCR8 antibody with Fc-DLE, Fc-DE, Fc-Afucosylated, Fc-Asymmetric and Fc-WT (same Fab part was used). ADCC activity was determined by Jurkat hCD16a NFAT or Jurkat hCD16a hCD32b NFAT effecter cell, incubated with CHO-K1 hCCR8 cell. The results (FIG. 14A) showed that anti-hCCR8 antibodies with Fc-DLE, Fc-DE, Fc-Afucosylated, Fc-Asymmetric induced potent ADCC activity, compared with WT type hIgG1. While in Jurkat hCD16a hCD32b cell coculture system where the inhibitory receptor was expressed, anti-hCCR8 antibodies with Fc-DLE, Fc-Afucosylated and Fc-Asymmetric showed stronger ADCC activity compared to Fc-DE and WT type hlgG1. This result was consistent with the A/I ratio (FIG. 14B).

Additionally, antibodies mediated killing against CCR8 expressing HEK293 cells were tested. PBMC from healthy donor co-incubated with HEK293 hCCR8 cell (ratio of PBMC to target cell is 50:1) were treated with serial dilutions of anti-CCR8 antibodies with Fc-DLE, Fc-DE, Fc-Afucosylated, Fc-WT and hlgG1 DLE control at 37° C. overnight. Cell lysis was determined by the released LDH level in the supernatant after co-culture. The results showed that anti-hCCR8 antibody with engineered Fc especially with DLE mutation mediated most potent killing of CCR8-expressing target cells (FIG. 14C).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An antibody or antigen-binding fragment thereof which has specificity to the human C-C motif chemokine receptor 8 (CCR8) protein and comprises a heavy chain variable region (VH) comprising a VH CDR1, a VH CDR2 and a VH CDR3, and a light chain variable region (VL) comprising a VL CDR1, a VL CDR2, and a VL CDR3, wherein:
(a) the VH CDR1 comprises the amino acid sequence of SEQ ID NO: 61;
the VH CDR2 comprises the amino acid sequence of SEQ ID NO: 62;
the VH CDR3 comprises the amino acid sequence of SEQ ID NO: 63 or 39;
the VL CDR1 comprises the amino acid sequence of SEQ ID NO: 64;
the VL CDR2 comprises the amino acid sequence of SEQ ID NO: 52; and
the VL CDR3 comprises the amino acid sequence of SEQ ID NO: 57,
(b) the VH CDR1 comprises the amino acid sequence of SEQ ID NO: 22;
the VH CDR2 comprises the amino acid sequence of SEQ ID NO: 58;
the VH CDR3 comprises the amino acid sequence of SEQ ID NO: 35, 36, 37 or 38;
the VL CDR1 comprises the amino acid sequence of SEQ ID NO: 59;
the VL CDR2 comprises the amino acid sequence of SEQ ID NO: 49, 50 or 51; and
the VL CDR3 comprises the amino acid sequence of SEQ ID NO: 60, or
(c) the VH CDR1 comprises the amino acid sequence of SEQ ID NO: 21;
the VH CDR2 comprises the amino acid sequence of SEQ ID NO: 25, 65 or 66;
the VH CDR3 comprises the amino acid sequence of SEQ ID NO: 34;
the VL CDR1 comprises the amino acid sequence of SEQ ID NO: 43;
the VL CDR2 comprises the amino acid sequence of SEQ ID NO: 48; and
the VL CDR3 comprises the amino acid sequence of SEQ ID NO: 53.

2. The antibody or antigen-binding fragment thereof of claim 1, wherein:
(a) the VH CDR1 comprises the amino acid sequence of SEQ ID NO: 61;
the VH CDR2 comprises the amino acid sequence of SEQ ID NO: 62;
the VH CDR3 comprises the amino acid sequence of SEQ ID NO: 63 or 39;
the VL CDR1 comprises the amino acid sequence of SEQ ID NO: 64;
the VL CDR2 comprises the amino acid sequence of SEQ ID NO: 52; and
the VL CDR3 comprises the amino acid sequence of SEQ ID NO: 57.

3. The antibody or antigen-binding fragment thereof of claim 2, wherein:
the VH CDR1 comprises the amino acid sequence of SEQ ID NO: 61;
the VH CDR2 comprises the amino acid sequence of SEQ ID NO: 62;
the VH CDR3 comprises the amino acid sequence of SEQ ID NO: 63;
the VL CDR1 comprises the amino acid sequence of SEQ ID NO: 64;
the VL CDR2 comprises the amino acid sequence of SEQ ID NO: 52; and
the VL CDR3 comprises the amino acid sequence of SEQ ID NO: 57.

4. The antibody or antigen-binding fragment thereof of claim 2, wherein the VH CDR1, VH CDR2, VH CDR3, VL CDR1, VL CDR2 and VL CDR3 comprise, respectively, the amino acid sequences of:
SEQ ID NO: 23, 31, 39, 46, 52 and 57;
SEQ ID NO: 23, 31, 39, 72, 52 and 57;
SEQ ID NO: 23, 31, 39, 73, 52 and 57;
SEQ ID NO: 24, 32, 40, 47, 52 and 57;
SEQ ID NO: 24, 32, 40, 74, 52 and 57;
SEQ ID NO: 24, 32, 40, 75, 52 and 57;
SEQ ID NO: 24, 32, 41, 47, 52 and 57;
SEQ ID NO: 24, 32, 41, 74, 52 and 57;
SEQ ID NO: 24, 32, 41, 75, 52 and 57;
SEQ ID NO: 24, 33, 42, 46, 52 and 57;
SEQ ID NO: 24, 33, 42, 72, 52 and 57; or
SEQ ID NO: 24, 33, 42, 73, 52 and 57.

5. The antibody or antigen-binding fragment thereof of claim 2, wherein the VH CDR1, VH CDR2, VH CDR3, VL CDR1, VL CDR2 and VL CDR3 comprise, respectively, the amino acid sequences of SEQ ID NO: 24, 32, 41, 74, 52 and 57.

6. The antibody or antigen-binding fragment thereof of claim 5, wherein the VH comprises an amino acid sequence selected from the group consisting of SEQ ID NO: 76-79 and the VL comprises an amino acid sequence selected from the group consisting of SEQ ID NO: 92-95.

7. The antibody or antigen-binding fragment thereof of claim 2, wherein the VH CDR1, VH CDR2, VH CDR3, VL CDR1, VL CDR2 and VL CDR3 comprise, respectively, the amino acid sequences of SEQ ID NO: 24, 32, 41, 47, 52 and 57.

8. The antibody or antigen-binding fragment thereof of claim 7, wherein the VH comprises an amino acid sequence selected from the group consisting of SEQ ID NO: 17 and 76-79 and the VL comprises an amino acid sequence selected from the group consisting of SEQ ID NO: 18 and 80-83.

9. The antibody or antigen-binding fragment thereof of claim 2, wherein the VH CDR1, VH CDR2, VH CDR3, VL CDR1, VL CDR2 and VL CDR3 comprise, respectively, the amino acid sequences of SEQ ID NO: 24, 33, 42, 72, 52 and 57.

10. The antibody or antigen-binding fragment thereof of claim 9, wherein the VH comprises an amino acid sequence selected from the group consisting of SEQ ID NO: 84-87 and the VL comprises an amino acid sequence selected from the group consisting of SEQ ID NO: 96-99.

11. The antibody or antigen-binding fragment thereof of claim 2, wherein the VH CDR1, VH CDR2, VH CDR3, VL CDR1, VL CDR2 and VL CDR3 comprise, respectively, the amino acid sequences of SEQ ID NO: 24, 33, 42, 46, 52 and 57.

12. The antibody or antigen-binding fragment thereof of claim 11, wherein the VH comprises an amino acid sequence selected from the group consisting of SEQ ID NO: 19 and 84-87 and the VL comprises an amino acid sequence selected from the group consisting of SEQ ID NO: 20 and 88-91, preferably wherein the VH comprises the amino acid sequence of SEQ ID NO: 85 and the VL comprises the amino acid sequence of SEQ ID NO: 98.

13. The antibody or antigen-binding fragment thereof of claim 4, wherein the VH comprises the amino acid sequence of SEQ ID NO: 13 and the VL comprises the amino acid sequence of SEQ ID NO: 14.

14. The antibody or antigen-binding fragment thereof of claim 4, wherein the VH comprises the amino acid sequence of SEQ ID NO: 15 and the VL comprises the amino acid sequence of SEQ ID NO: 16.

15. The antibody or antigen-binding fragment thereof of claim 1, wherein:
(b) the VH CDR1 comprises the amino acid sequence of SEQ ID NO: 22;
the VH CDR2 comprises the amino acid sequence of SEQ ID NO: 58;
the VH CDR3 comprises the amino acid sequence of SEQ ID NO: 35, 36, 37 or 38;
the VL CDR1 comprises the amino acid sequence of SEQ ID NO: 59;
the VL CDR2 comprises the amino acid sequence of SEQ ID NO: 49, 50 or 51; and
the VL CDR3 comprises the amino acid sequence of SEQ ID NO: 60,

16. The antibody or antigen-binding fragment thereof of claim 15, wherein the VH CDR1, VH CDR2, VH CDR3, VL CDR1, VL CDR2 and VL CDR3 comprise, respectively, the amino acid sequences of:
SEQ ID NO: 22, 26, 35, 44, 49 and 54;
SEQ ID NO: 22, 67, 35, 44, 49 and 54;
SEQ ID NO: 22, 27, 36, 45, 50 and 55;
SEQ ID NO: 22, 68, 36, 45, 50 and 55;
SEQ ID NO: 22, 28, 37, 44, 49 and 54;
SEQ ID NO: 22, 69, 37, 44, 49 and 54;
SEQ ID NO: 22, 29, 36, 45, 50 and 55;
SEQ ID NO: 22, 70, 36, 45, 50 and 55;
SEQ ID NO: 22, 30, 38, 44, 51 and 56; or
SEQ ID NO: 22, 71, 38, 44, 51 and 56.

17. The antibody or antigen-binding fragment thereof of claim 16, wherein the VH and VL comprise, respectively, the amino acid sequences of:
SEQ ID NO: 3 and 4;
SEQ ID NO: 5 and 6;
SEQ ID NO: 7 and 8;
SEQ ID NO: 9 and 10; or
SEQ ID NO: 11 and 12.

18. The antibody or antigen-binding fragment thereof of claim 1, wherein the VH CDR1, VH CDR2, VH CDR3, VL CDR1, VL CDR2 and VL CDR3 comprise, respectively, the amino acid sequences of:
SEQ ID NO: 21, 25, 34, 43, 48 and 53;
SEQ ID NO: 21, 65, 34, 43, 48 and 53; or
SEQ ID NO: 21, 66, 34, 43, 48 and 53.

19. The antibody or antigen-binding fragment thereof of claim 18, wherein the VH comprises the amino acid sequence of SEQ ID NO: 1 and the VL comprises the amino acid sequence of SEQ ID NO: 2.

20-32. (canceled)

33. A method of treating cancer or an inflammatory condition in a patient in need thereof, comprising administering to the patient an effective amount of the antibody or antigen-binding fragment thereof of claim 1.

34-37. (canceled)
